(19) United States
(12) Patent Application Publication
(10) Pub. No.: US 2024/0032844 A1
(43) Pub. Date: Feb. 1, 2024

(54) ATRIAL ELECTRICAL HETEROGENEITY

(71) Applicant: Medtronic, Inc., Minneapolis, MN (US)

(72) Inventor: Subham Ghosh, Blaine, MN (US)

(21) Appl. No.: 18/220,911

(22) Filed: Jul. 12, 2023

Related U.S. Application Data
(60) Provisional application No. 63/392,910, filed on Jul. 28, 2022.

(51) Int. Cl. A61B 5/353; A61B 5/00

(52) U.S. Cl. CPC A61B 5/353; A61B 5/6823; A61B 5/7203

(57) ABSTRACT

Systems and methods of generating atrial electrical heterogeneity using electrical activity monitored using a plurality of external electrodes are described herein. The atrial electrical heterogeneity may include P-wave electrical heterogeneity. The atrial electrical heterogeneity generated from electrical activity monitored during atrial pacing therapy may be used to evaluate and adjust atrial pacing therapy. The atrial electrical heterogeneity generated from electrical activity monitored during intrinsic activation may be used to evaluate a patient's cardiac condition.

### RELATED APPLICATIONS

This application claims the benefit of U.S. Provisional Application Ser. No. 63/392,910, filed Jul. 28, 2022, the entire contents of each of which are incorporated herein by reference.

### TECHNICAL FIELD

The disclosure herein relates to systems and methods for use in determining atrial electrical heterogeneity.

### BACKGROUND

During normal sinus rhythm (NSR), the heart beat is regulated by electrical signals produced by the sino-atrial (SA) node located in the right atrial wall. Each atrial depolarization signal produced by the SA node spreads across the atria, causing the depolarization and contraction of the atria, and arrives at the atrioventricular (A-V) node. The A-V node responds by propagating a ventricular depolarization signal through the bundle of His of the ventricular septum and thereafter to the bundle branches and the Purkinje muscle fibers of the right and left ventricles.

Atrial tachyarrhythmia includes the disorganized form of atrial fibrillation and varying degrees of organized atrial tachycardia, including atrial flutter. Atrial fibrillation (AF) occurs because of multiple focal triggers in the atrium or because of changes in the substrate of the atrium causing heterogeneities in conduction through different regions of the atria. The ectopic triggers can originate anywhere in the left or right atrium or pulmonary veins. The AV node will be bombarded by frequent and irregular atrial activations but will only conduct a depolarization signal when the AV node is not refractory. The ventricular cycle lengths will be irregular and will depend on the different states of refractoriness of the AV-node.

Patients with atrial dyssynchrony have wide P-waves and slower atrial conduction which may result in delayed atrial kick and lessened contribution of atrial kick to filling which can compromise ventricular function (heart failure). Atrial dyssynchrony may also be a cause for atrial arrhythmias and atrial fibrillation. Atrial pacing therapy like targeting Bachmann's bundle for synchronized atrial activation or other means of multi-site atrial stimulation may help restore atrial synchrony and lead to better outcomes in these patients including bettering heart failure symptoms and potentially reducing burden of atrial arrhythmias. Therefore, there is a growing interest in a method and apparatus for improving determination of a desired location or locations from which to delivery pacing therapy from within the atrium that results in overall improvement of bi-atrial synchrony.

### SUMMARY

The illustrative systems and methods described herein may be configured to assist a user (e.g., a physician) in evaluating and configuring cardiac therapy (e.g., cardiac therapy being performed on a patient during and/or after implantation of cardiac therapy apparatus). In one or more embodiments, the systems and methods may be described as being noninvasive. For example, in some embodiments, the systems and methods may not need, or include, implantable devices such as leads, probes, sensors, catheters, implantable electrodes, etc. to monitor, or acquire, a plurality of cardiac signals from tissue of the patient for use in evaluating and configuring the cardiac therapy being delivered to the patient. Instead, the systems and methods may use electrical measurements taken noninvasively using, e.g., a plurality of external electrodes attached to the skin of a patient about the patient's torso.

The illustrative systems and methods may include the use of an external electrode apparatus, or an ECG belt, that is applied to the torso of a patient to determine atrial electrical heterogeneity to, e.g., evaluate and configure (e.g., optimize) pacing therapies such as atrial pacing therapies. Atrial pacing may include, for example, atrial septal pacing, Bachmann Bundle pacing, left atrial pacing, and biatrial pacing. Additionally, determination of atrial electrical heterogeneity may also be useful for evaluation of risk-stratifying patients at risk of arrhythmias like atrial fibrillation. Surface ECG P-waves are often small and noisy and are difficult to accurately measure timing fiducials on P-waves. The illustrative systems and methods may be described as improved systems and methods of quantifying atrial electrical heterogeneity from multi-electrode ECG signals.

One illustrative system may include a plurality of external electrodes to be located proximate the skin of the torso of a patient to measure electrical activity from tissue of the patient and a computing apparatus operably coupled to the plurality of external electrodes and comprising processing circuitry. The computing apparatus may be configured to obtain external electrical activity measured from tissue of a patient and generate a P-wave electrical heterogeneity based on the obtained electrical activity.

One illustrative method may include obtaining external electrical activity measured from tissue of a patient using a plurality of external electrodes and generating a P-wave electrical heterogeneity based on the obtained electrical activity.

The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is a diagram of an illustrative system including electrode apparatus, display apparatus, and computing apparatus.

FIGS. 2-3 are diagrams of illustrative external electrode apparatus for measuring torso-surface potentials.

FIG. 4 provides a block diagram of an illustrative method of generation of atrial electrical heterogeneity.

FIG. 5A illustrates monitored electrical activity during intrinsic activation and a generated P-wave electrical heterogeneity therefrom.

FIG. 5B illustrates monitored electrical activity during Bachman bundle pacing and a generated P-wave electrical heterogeneity therefrom.

FIG. 6A illustrates generated P-wave electrical heterogeneity during intrinsic activation and during Bachman bundle pacing for three different patients.

FIG. 6B illustrates generated P-wave duration during intrinsic activation and during Bachman bundle pacing for three different patients.

FIG. 7 is a diagram of an illustrative system including an illustrative implantable medical device (IMD).

FIG. 8A is a diagram of the illustrative IMD of FIG. 7.

FIG. 8B is a diagram of an enlarged view of a distal end of the electrical lead disposed in the left ventricle of FIG. 8A.

FIG. 9A is a block diagram of an illustrative IMD, e.g., of the systems of FIGS. 7-8.

FIG. 9B is another block diagram of an illustrative IMD (e.g., an implantable pulse generator) circuitry and associated leads employed in the systems of FIGS. 7-8).

### DETAILED DESCRIPTION

In the following detailed description of illustrative embodiments, reference is made to the accompanying figures of the drawing which form a part hereof, and in which are shown, by way of illustration, specific embodiments which may be practiced. It is to be understood that other embodiments may be utilized, and structural changes may be made without departing from (e.g., still falling within) the scope of the disclosure presented hereby.

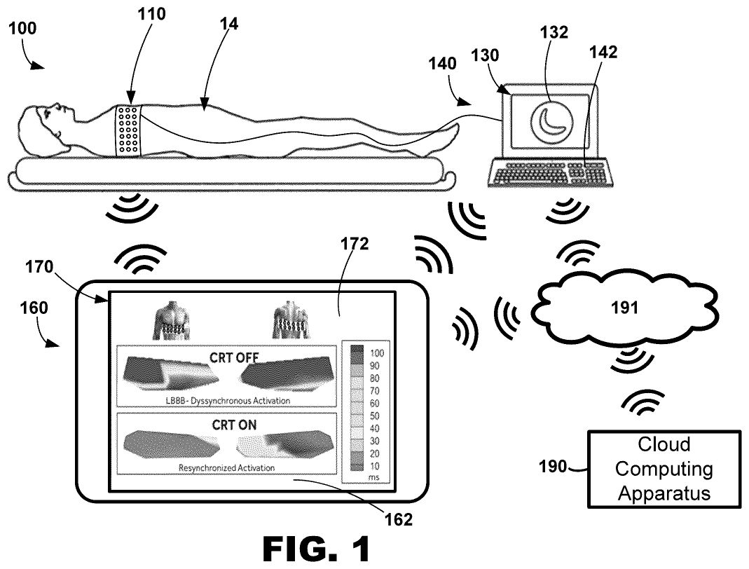

Illustrative systems, methods, and devices shall be described with reference to FIGS. 1-9. It will be apparent to one skilled in the art that elements or processes from one embodiment may be used in combination with elements or processes of the other embodiments, and that the possible embodiments of such systems, methods, and devices using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

A plurality of electrocardiogram (ECG) signals (e.g., torso-surface potentials) may be measured, or monitored, using a plurality of external electrodes positioned about the surface, or skin, of a patient. The ECG signals may be used to evaluate and configure cardiac therapy such as, e.g., atrial pacing therapy, cardiac resynchronization therapy (CRT), etc. As described herein, the ECG signals may be gathered or obtained noninvasively since, e.g., implantable electrodes may not be used to measure the ECG signals. Further, the ECG signals may be used to determine cardiac electrical activation times, which may be used to generate various metrics (e.g., atrial electrical heterogeneity information, P-wave electrical heterogeneity, a maximum or largest distribution metric of P-wave electrical activity, etc.) that may be used automatically by the illustrative systems, devices, and methods and/or by a user (e.g., physician) to optimize one or more settings, or parameters, of cardiac therapy (e.g., atrial pacing therapy, CRT, etc.).

Various illustrative systems, methods, devices, and graphical user interfaces provided thereby may be configured to use electrode apparatus including external electrodes, display apparatus, and computing apparatus to noninvasively assist a user (e.g., a physician) in the evaluation of cardiac health and/or the configuration (e.g., optimization) of cardiac therapy. An illustrative system 100 including electrode apparatus 110 and computing apparatus is depicted in FIG. 1.

The computing apparatus may include, among other things, a local computing device 140, a remote computing device 160, and a cloud computing device 190. It is to be understood that the computing apparatus may include any one of the local computing device 140, the remote computing device 160, and the cloud computing device 190 or any combination of the local computing device 140, the remote computing device 160, and the cloud computing device 190 operating in conjunction with each other.

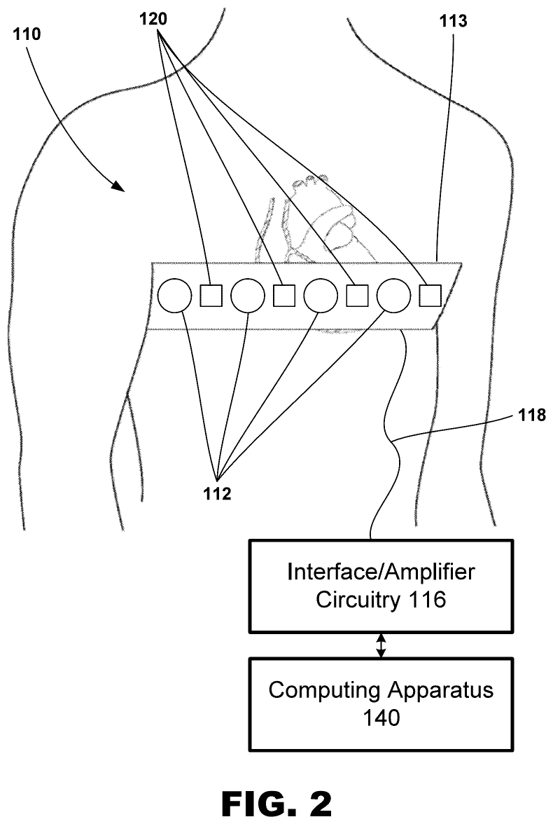

The electrode apparatus 110 as shown includes a plurality of electrodes incorporated, or included, within a band wrapped around the chest, or torso, of a patient 14. The electrode apparatus 110 is operatively coupled to the local computing device 140 (e.g., through one or wired electrical connections, wirelessly, etc.) to provide electrical signals from each of the electrodes to the local computing device 140 for analysis, evaluation, etc. Illustrative electrode apparatus may be described in U.S. Pat. No. 9,320,446 entitled “Bioelectric Sensor Device and Methods” filed Mar. 27, 2014, and issued on Mar. 26, 2016, which is incorporated herein by reference in its entirety. Further, illustrative electrode apparatus 110 will be described in more detail in reference to FIGS. 2-3.

Although not described herein, the illustrative system 100 may further include imaging apparatus. The imaging apparatus may be any type of imaging apparatus configured to image, or provide images of, at least a portion of the patient in a noninvasive manner. For example, the imaging apparatus may not use any components or parts that may be located within the patient to provide images of the patient except noninvasive tools such as contrast solution. It is to be understood that the illustrative systems, methods, and interfaces described herein may further use imaging apparatus to provide noninvasive assistance to a user (e.g., a physician) to locate, or place, one or more pacing electrodes proximate the patient's heart in conjunction with the configuration of cardiac therapy.

For example, the illustrative systems, methods, and devices, may provide image guided navigation that may be used to navigate leads including electrodes, leadless electrodes, wireless electrodes, catheters, etc., within the patient's body while also providing noninvasive cardiac therapy configuration including determination of an effective, or optimal, pre-excitation intervals such as A-V and V-V intervals, etc. Illustrative systems, methods, and devices that use imaging apparatus and/or electrode apparatus may be described in U.S. Pat. App. Pub. No. 2014/0371832 to Ghosh published on Dec. 18, 2014, U.S. Pat. App. Pub. No. 2014/0371833 to Ghosh et al. published on Dec. 18, 2014, U.S. Pat. App. Pub. No. 2014/0323892 to Ghosh et al. published on Oct. 30, 2014, U.S. Pat. App. Pub. No. 2014/0323882 to Ghosh et al. published on Oct. 20, 2014, each of which is incorporated herein by reference in its entirety.

Illustrative imaging apparatus may be configured to capture x-ray images and/or any other alternative imaging modality. For example, the imaging apparatus may be configured to capture images, or image data, using isocentric fluoroscopy, bi-plane fluoroscopy, ultrasound, computed tomography (CT), multi-slice computed tomography (MSCT), magnetic resonance imaging (MRI), high frequency ultrasound (HIFU), optical coherence tomography (OCT), intra-vascular ultrasound (IVUS), two dimensional (2D) ultrasound, three dimensional (3D) ultrasound, four dimensional (4D) ultrasound, intraoperative CT, intraoperative MRI, etc. Further, it is to be understood that the imaging apparatus may be configured to capture a plurality of consecutive images (e.g., continuously) to provide video frame data. In other words, a plurality of images taken over time using the imaging apparatus may provide video frame, or motion picture, data. An exemplary system that employs ultrasound can be found in U.S. Pat. App. Pub. No. 2017/0303840 to Stadler et al. published on Oct. 26, 2017, which is incorporated by reference in its entirety. Additionally, the images may also be obtained and displayed in two, three, or four dimensions. In more advanced forms, four-dimensional surface rendering of the heart or other regions of the body may also be achieved by incorporating heart data or other soft tissue data from a map or from pre-operative image data captured by MRI, CT, or echocardiography modalities. Image datasets from hybrid modalities, such as positron emission tomography (PET) combined with CT, or single photon emission computer tomography (SPECT) combined with CT, could also provide functional image data superimposed onto anatomical data, e.g., to be used to navigate implantable apparatus to target locations within the heart or other areas of interest.

Systems and/or imaging apparatus that may be used in conjunction with the illustrative systems, methods, and devices described herein are described in U.S. Pat. App. Pub. No. 2005/0008210 to Evron et al. published on Jan. 13, 2005, U.S. Pat. App. Pub. No. 2006/0074285 to Zarkh et al. published on Apr. 6, 2006, U.S. Pat. No. 8,731,642 to Zarkh et al. issued on May 20, 2014, U.S. Pat. No. 8,861,830 to Brada et al. issued on Oct. 14, 2014, U.S. Pat. No. 6,980,675 to Evron et al. issued on Dec. 27, 2005, U.S. Pat. No. 7,286,866 to Okerlund et al. issued on Oct. 23, 2007, U.S. Pat. No. 7,308,297 to Reddy et al. issued on Dec. 11, 2011, U.S. Pat. No. 7,308,299 to Burrell et al. issued on Dec. 11, 2011, U.S. Pat. No. 7,321,677 to Evron et al. issued on Jan. 22, 2008, U.S. Pat. No. 7,346,381 to Okerlund et al. issued on Mar. 18, 2008, U.S. Pat. No. 7,454,248 to Burrell et al. issued on Nov. 18, 2008, U.S. Pat. No. 7,499,743 to Vass et al. issued on Mar. 3, 2009, U.S. Pat. No. 7,565,190 to Okerlund et al. issued on Jul. 21, 2009, U.S. Pat. No. 7,587,074 to Zarkh et al. issued on Sep. 8, 2009, U.S. Pat. No. 7,599,730 to Hunter et al. issued on Oct. 6, 2009, U.S. Pat. No. 7,613,500 to Vass et al. issued on Nov. 3, 2009, U.S. Pat. No. 7,742,629 to Zarkh et al. issued on Jun. 22, 2010, U.S. Pat. No. 7,747,047 to Okerlund et al. issued on Jun. 29, 2010, U.S. Pat. No. 7,778,685 to Evron et al. issued on Aug. 17, 2010, U.S. Pat. No. 7,778,686 to Vass et al. issued on Aug. 17, 2010, U.S. Pat. No. 7,813,785 to Okerlund et al. issued on Oct. 12, 2010, U.S. Pat. No. 7,996,063 to Vass et al. issued on Aug. 9, 2011, U.S. Pat. No. 8,060,185 to Hunter et al. issued on Nov. 15, 2011, and U.S. Pat. No. 8,401,616 to Verard et al. issued on Mar. 19, 2013, each of which is incorporated herein by reference in its entirety.

The local computing device 140, the remote computing device 160, and the cloud computing device 190 may be configured to monitor (e.g., using the electrode apparatus), generate, and analyze data such as, e.g., electrical signals (e.g., electrocardiogram data), P-wave signals, QRS complexes, atrial electrical heterogeneity, P-wave electrical heterogeneity, electrical activation times, electrical heterogeneity information, etc. For example, one cardiac cycle, or one heartbeat, of a plurality of cardiac cycles, or heartbeats, represented by the electrical signals collected or monitored by the electrode apparatus 110 may be analyzed and evaluated for one or more metrics including atrial electrical heterogeneity, P-wave electrical heterogeneity, and activation times that may be pertinent to the therapeutic nature of one or more parameters related to cardiac therapy such as, e.g., pacing parameters, pacing mode, pacing vector, lead location, etc. More specifically, for example, a P-wave of a single cardiac cycle may be evaluated for one or more metrics such as, e.g., P-wave onset, P-wave offset, QRS onset, QRS offset, atrial electrical heterogeneity, P-wave electrical heterogeneity, a distribution metric of each signal, a maximum or largest distribution metric of a plurality of signals, a standard deviation of each signal with a P-wave window or lookback window from QRS onset, a maximum or largest standard deviation of a plurality of signals with a P-wave window or lookback window from QRS onset, QRS peak, electrical activation times referenced to earliest activation time, electrical heterogeneity information (EHI), other statistical measures of central tendency (e.g., median or mode), dispersion (e.g., mean deviation, standard deviation, variance, interquartile deviations, range, etc.), etc. Further, each of the one or more metrics may be location specific. For example, some metrics may be computed from signals recorded, or monitored, from electrodes positioned about a selected area of the patient such as, e.g., the left side of the patient, the right side of the patient, etc. Additionally, the local computing device 140 and the remote computing device 160 may each include display apparatus 130, 170, respectively, that may be configured to display such data.

In at least one embodiment, each of the local computing device 140, the remote computing device 160, and the cloud computing device 190 may be a server, a personal computer, a tablet computer, a mobile device, and a cellular telephone. The local computing device 140 may be configured to receive input from input apparatus 142 (e.g., a keyboard) and transmit output to the display apparatus 130, and the remote computing device 160 may be configured to receive input from input apparatus 162 (e.g., a touchscreen) and transmit output to the display apparatus 170. The local computing device 140, the remote computing device 160, and the cloud computing device 190 may include data storage that may allow for access to processing programs or routines and/or one or more other types of data, e.g., for analyzing a plurality of electrical signals captured by the electrode apparatus 110, for determining P-wave onsets, P-wave offset, QRS onsets, QRS offsets, distribution metrics such as standard deviations, medians, modes, averages, peaks or maximum values, valleys or minimum values in such electrical signals, for determining atrial electrical heterogeneity such as P-wave electrical heterogeneity, for determining electrical activation times, for configuring one or more pacing parameters, or settings, such as, e.g., pacing rate, atrial pacing rate, ventricular pacing rate, A-V interval, V-V interval, pacing amplitude, pacing pulse width, pacing vector, multipoint pacing vector (e.g., left ventricular vector quad lead), pacing voltage, pacing configuration (e.g., biventricular pacing, right ventricle only pacing, left ventricle only pacing, etc.), for driving graphical user interfaces used to configured one or more pacing parameters, for arrhythmia detection and treatment, for patient risk stratification, etc.

The local computing device 140 may be operatively coupled to the input apparatus 142 and the display apparatus 130 to, e.g., transmit data to and from each of the input apparatus 142 and the display apparatus 130, and the remote computing device 160 may be operatively coupled to the input apparatus 162 and the display apparatus 170 to, e.g., transmit data to and from each of the input apparatus 162 and the display apparatus 170. For example, the local computing device 140 and the remote computing device 160 may be electrically coupled to the input apparatus 142, 162 and the display apparatus 130, 170 using, e.g., analog electrical connections, digital electrical connections, wireless connections, bus-based connections, network-based connections, internet-based connections, etc. As described further herein, a user may provide input to the input apparatus 142, 162 to view and/or select one or more pieces of configuration information related to the cardiac therapy delivered by cardiac therapy apparatus such as, e.g., an implantable medical device.

Although as depicted the input apparatus 142 is a keyboard and the input apparatus 162 is a touchscreen, it is to be understood that the input apparatus 142, 162 may include any apparatus capable of providing input to the local computing device 140 and the computing device 160 to perform the functionality, methods, and/or logic described herein. For example, the input apparatus 142, 162 may include a keyboard, a mouse, a trackball, a touchscreen (e.g., capacitive touchscreen, a resistive touchscreen, a multi-touch touchscreen, etc.), etc. Likewise, the display apparatus 130, 170 may include any apparatus capable of displaying information to a user, such as a graphical user interface 132, 172 including electrode status information, graphical maps of electrical activation, a plurality of signals for the external electrodes over one or more heartbeats, QRS complexes, various pacing parameters (e.g., including selected pacing parameters as determined using the illustrative systems, methods, and devices described herein, atrial electrical heterogeneity, P-wave electrical heterogeneity, electrical heterogeneity information (EHI), textual instructions, graphical depictions of anatomy of a human heart, images or graphical depictions of the patient's heart, graphical depictions of locations of one or more electrodes, graphical depictions of a human torso, images or graphical depictions of the patient's torso, graphical depictions or actual images of implanted electrodes and/or leads, etc. Further, the display apparatus 130, 170 may include a liquid crystal display, an organic light-emitting diode screen, a touchscreen, a cathode ray tube display, etc.

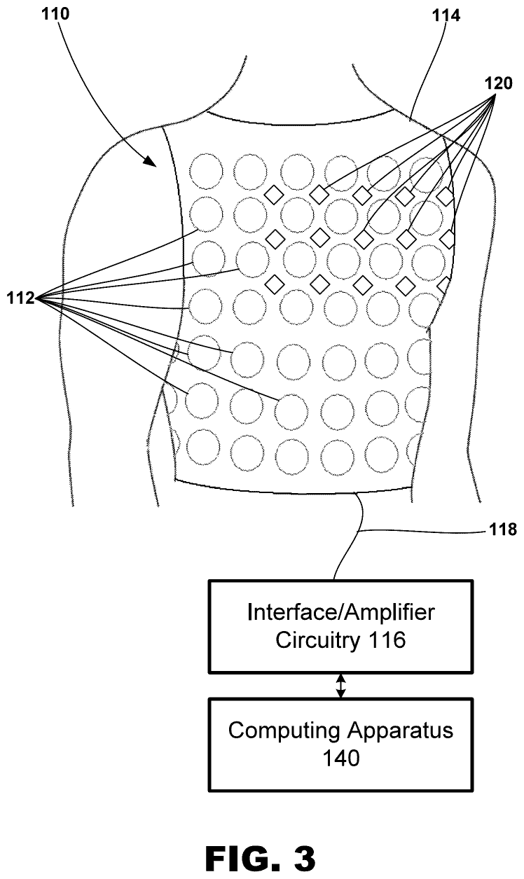

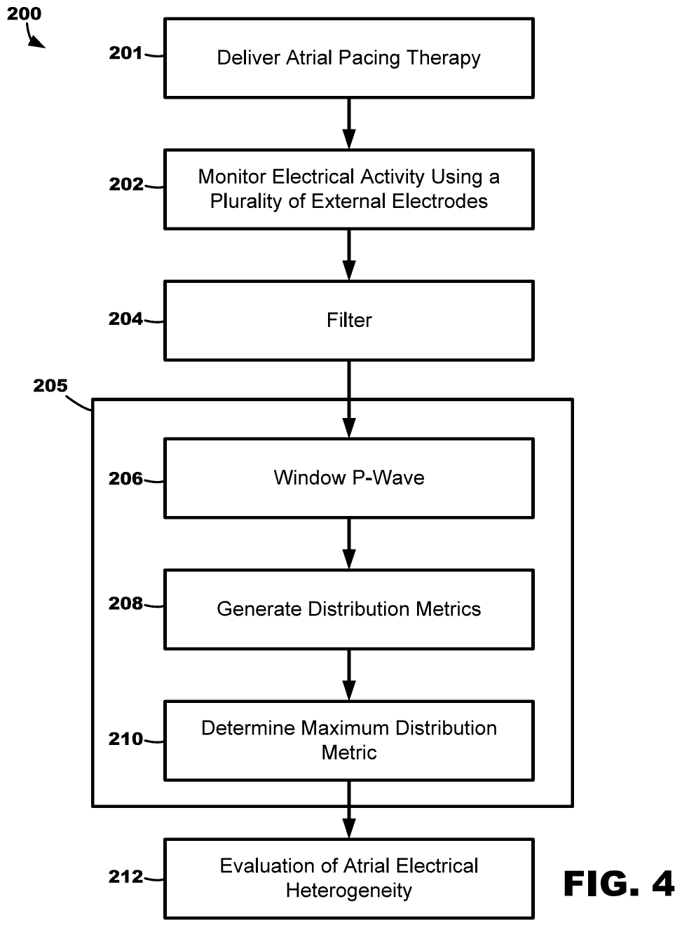

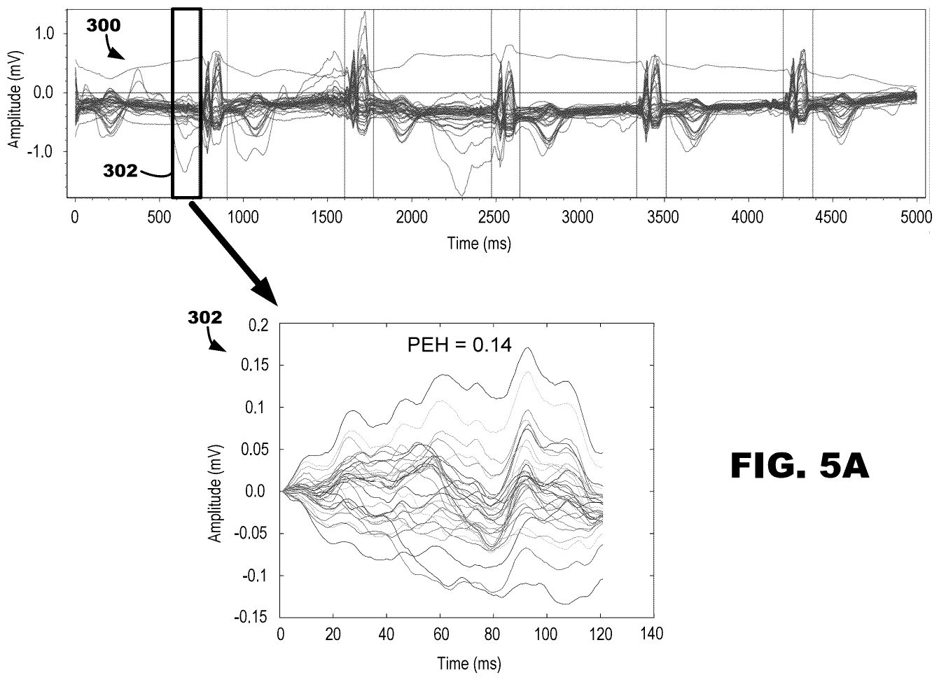

The processing programs or routines stored and/or executed by the local computing device 140, the remote computing device 160, and the cloud computing device 190 may include programs or routines for computational mathematics, matrix mathematics, decomposition algorithms, compression algorithms (e.g., data compression algorithms), calibration algorithms, image construction algorithms, signal processing algorithms (e.g., various filtering algorithms, Fourier transforms, fast Fourier transforms, etc.), standardization algorithms, comparison algorithms, vector mathematics, or any other processing used to implement one or more illustrative methods and/or processes described herein. Data stored and/or used by the local computing device 140, the remote computing device 160, and the cloud computing device 190 may include, for example, electrical signal/waveform data from the electrode apparatus 110 (e.g., a plurality of QRS complexes), electrical activation times from the electrode apparatus 110, cardiac sound/signal/waveform data from acoustic sensors, graphics (e.g., graphical elements, icons, buttons, windows, dialogs, pull-down menus, graphic areas, graphic regions, 3D graphics, etc.), graphical user interfaces, results from one or more processing programs or routines employed according to the disclosure herein (e.g., electrical signals, electrical heterogeneity information, atrial electrical heterogeneity, P-wave electrical heterogeneity, etc.), or any other data that may be used for carrying out the one and/or more processes or methods described herein. In one or more embodiments, one or more of the local computing device 140, the remote computing device 160, and the cloud computing device 190 may be configured to select and/or configure atrial pacing therapy and/or evaluate a patient's condition based on monitored and generated data from, for example, the electrode apparatus 110 as will be described further herein with respect to FIGS. 4-5. In particular, for example, one or more of the local computing device 140, the remote computing device 160, and the cloud computing device 190 may monitor electrical activity using the electrode apparatus 110 and generate atrial electrical heterogeneity such as P-wave electrical heterogeneity therefrom during delivery of pacing therapy a plurality of different configurations or settings. For instance, one or more of the local computing device 140, the remote computing device 160, and the cloud computing device 190 may generate atrial electrical heterogeneity from electrical activity measured during delivery of pacing therapy at a plurality of different atrial pacing vectors, and then may determine which of the plurality of different atrial pacing vectors is desired (e.g., optimal), which may be selected. Further, one or more of the local computing device 140, the remote computing device 160, and the cloud computing device 190 may generate atrial electrical heterogeneity from electrical activity measured during delivery of atrial pacing therapy configured, and then may determine the atrial pacing therapy is acceptable or desired (e.g., optimal). Still further, one or more of the local computing device 140, the remote computing device 160, and the cloud computing device 190 may generate atrial electrical heterogeneity from electrical activity measured during delivery of atrial pacing therapy at a plurality of settings (e.g., timing, pulse width, amplitude, etc.), and then may determine which of the plurality of different settings is acceptable or desired (e.g., optimal).

In one or more embodiments, the illustrative systems, methods, devices, and interfaces may be implemented using one or more computer programs executed on programmable computers, such as computers that include, for example, processing capabilities, data storage (e.g., volatile or non-volatile memory and/or storage elements), input devices, and output devices. Program code and/or logic described herein may be applied to input data to perform functionality described herein and generate desired output information. The output information may be applied as input to one or more other systems, methods, devices, and/or interfaces as described herein or as would be applied in a known fashion.

The one or more programs used to implement the systems, methods, devices, and/or interfaces described herein may be provided using any programmable language, e.g., a high-level procedural and/or object orientated programming language that is suitable for communicating with a computer. Any such programs may, for example, be stored on any suitable device, e.g., a storage media, that is readable by a general or special purpose program running on a computer (e.g., including processing circuitry or processing apparatus) for configuring and operating the computer when the suitable device is read for performing the procedures described herein. In other words, at least in one embodiment, the illustrative systems, methods, devices, and interfaces may be implemented using a tangible computer readable storage medium, configured with a computer program, where the storage medium so configured causes the computer to operate in a specific and predefined manner to perform functions described herein. Further, in at least one embodiment, the illustrative systems, methods, devices, and interfaces may be described as being implemented by logic (e.g., object code) encoded in one or more non-transitory media that includes code for execution and, when executed by a processor or processing circuitry, is operable to perform operations such as the methods, processes, and/or functionality described herein.

The local computing device 140, the remote computing device 160, and the cloud computing device 190 may be, for example, any fixed or mobile computer system (e.g., a controller, a microcontroller, a personal computer, minicomputer, tablet computer, etc.) including processing circuitry. The exact configurations of the local computing device 140, the remote computing device 160, and the cloud computing device 190 are not limiting, and essentially any device including processing circuitry and capable of providing suitable computing capabilities and control capabilities (e.g., signal analysis, mathematical functions such as medians, modes, averages, maximum value determination, minimum value determination, slope determination, minimum slope determination, maximum slope determination, graphics processing, etc.) may be used. As described herein, a digital file may be any medium (e.g., volatile or non-volatile memory, a CD-ROM, a punch card, magnetic recordable tape, etc.) containing digital bits (e.g., encoded in binary, trinary, etc.) that may be readable and/or writeable by the local computing device 140, the remote computing device 160, and the cloud computing device 190 described herein. Also, as described herein, a file in user-readable format may be any representation of data (e.g., ASCII text, binary numbers, hexadecimal numbers, decimal numbers, graphically, etc.) presentable on any medium (e.g., paper, a display, etc.) readable and/or understandable by a user.

In view of the above, it will be readily apparent that the functionality as described in one or more embodiments according to the present disclosure may be implemented in any manner as would be known to one skilled in the art. As such, the computer language, the computer system, or any other software/hardware which is to be used to implement the processes described herein shall not be limiting on the scope of the systems, processes, or programs (e.g., the functionality provided by such systems, processes, or programs) described herein.

The illustrative electrode apparatus 110 may be configured to measure body-surface potentials of a patient 14 and, more particularly, torso-surface potentials of a patient 14. As shown in FIG. 2, the illustrative electrode apparatus 110 may include a set, or array, of external electrodes 112, a strap 113, and interface/amplifier circuitry 116. The electrodes 112 may be attached, or coupled, to the strap 113 and the strap 113 may be configured to be wrapped around the torso of a patient 14 such that the electrodes 112 surround the patient's heart. As further illustrated, the electrodes 112 may be positioned around the circumference of a patient 14, including the posterior, lateral, posterolateral, anterolateral, and anterior locations of the torso of a patient 14.

The illustrative electrode apparatus 110 may be further configured to measure, or monitor, sounds from the patient 14. As shown in FIG. 2, the illustrative electrode apparatus 110 may include a set, or array, of acoustic sensors 120 attached, or coupled, to the strap 113. The strap 113 may be configured to be wrapped around the torso of a patient 14 such that the acoustic sensors 120 surround the patient's heart. As further illustrated, the acoustic sensors 120 may be positioned around the circumference of a patient 14, including the posterior, lateral, posterolateral, anterolateral, and anterior locations of the torso of a patient 14.

Further, the electrodes 112 and the acoustic sensors 120 may be electrically connected to interface/amplifier circuitry 116 via wired connection 118. The interface/amplifier circuitry 116 may be configured to amplify the signals from the electrodes 112 and the acoustic sensors 120 and provide the signals to one or more of the local computing device 140, the remote computing device 160, and the cloud computing device 190. Other illustrative systems may use a wireless connection to transmit the signals sensed by electrodes 112 and the acoustic sensors 120 to the interface/amplifier circuitry 116 and, in turn, to one or more of the local computing device 140, the remote computing device 160, and the cloud computing device 190, e.g., as channels of data. In one or more embodiments, the interface/amplifier circuitry 116 may be electrically coupled to the local computing device 140 using, e.g., analog electrical connections, digital electrical connections, wireless connections, bus-based connections, network-based connections, internet-based connections, etc.

Although in the example of FIG. 2 the electrode apparatus 110 includes a strap 113, in other examples any of a variety of mechanisms, e.g., tape or adhesives, may be employed to aid in the spacing and placement of electrodes 112 and the acoustic sensors 120. In some examples, the strap 113 may include an elastic band, strip of tape, or cloth. Further, in some examples, the strap 113 may be part of, or integrated with, a piece of clothing such as, e.g., a t-shirt or hospital gown. In other examples, the electrodes 112 and the acoustic sensors 120 may be placed individually on the torso of a patient 14. Further, in other examples, one or both of the electrodes 112 (e.g., arranged in an array) and the acoustic sensors 120 (e.g., also arranged in an array) may be part of, or located within, patches, vests, and/or other manners of securing the electrodes 112 and the acoustic sensors 120 to the torso of the patient 14. Still further, in other examples, one or both of the electrodes 112 and the acoustic sensors 120 may be part of, or located within, two sections of material or two patches. One of the two patches may be located on the anterior side of the torso of the patient 14 (to, e.g., monitor electrical signals representative of the anterior side of the patient's heart, measure surrogate cardiac electrical activation times representative of the anterior side of the patient's heart, monitor or measure sounds of the anterior side of the patient, etc.) and the other patch may be located on the posterior side of the torso of the patient 14 (to, e.g., monitor electrical signals representative of the posterior side of the patient's heart, measure surrogate cardiac electrical activation times representative of the posterior side of the patient's heart, monitor or measure sounds of the posterior side of the patient, etc.). And still further, in other examples, one or both of the electrodes 112 and the acoustic sensors 120 may be arranged in a top row and bottom row that extend from the anterior side of the patient 14 across the left side of the patient 14 to the posterior side of the patient 14. Yet still further, in other examples, one or both of the electrodes 112 and the acoustic sensors 120 may be arranged in a curve around the armpit area and may have an electrode/sensor-density that less dense on the right thorax that the other remaining areas.

The electrodes 112 may be configured to surround the heart of the patient 14 and record, or monitor, the electrical signals associated with the depolarization and repolarization of the heart after the signals have propagated through the torso of a patient 14. Each of the electrodes 112 may be used in a unipolar configuration to sense the torso-surface potentials that reflect the cardiac signals. The interface/amplifier circuitry 116 may also be coupled to a return or indifferent electrode (not shown) that may be used in combination with each electrode 112 for unipolar sensing.

In some examples, there may be about 12 to about 40 electrodes 112 and about 12 to about 40 acoustic sensors 120 spatially distributed around the torso of a patient. Other configurations may have more or fewer electrodes 112 and more or fewer acoustic sensors 120. For example, the number of electrodes 112 of the electrode apparatus 110 may be greater than or equal to 10, greater than or equal to 12, greater than or equal to 15, greater than or equal to 20, greater than or equal to 35, greater than or equal to 40, etc. and/or less than or equal to 80 electrodes, less than or equal to 70 electrodes, less than or equal to 60 electrodes, less than or equal to 50 electrodes, less than or equal to 45 electrodes, less than or equal to 38 electrodes, etc. In at least one embodiment, the electrode apparatus 110 includes 40 electrodes 112 with 20 of the electrodes 112 configured to be located on the posterior of the patient and the 20 of the electrodes 112 configured to be located on the anterior of the patient. In some embodiment, more electrodes 112 may be configured to be positioned on the anterior of the patient than the posterior of the patient or more electrodes 112 may be configured to be positioned on the posterior of the patient than the anterior of the patient. For example, in one embodiment, 25 electrodes 112 may be configured to be positioned on the anterior of the patient and 15 electrodes 112 may be configured to be positioned on the posterior of the patient. Additionally, in one embodiment, the electrodes 112 may be included as part of a 12-lead ECG apparatus. It is to be understood that the electrodes 112 and acoustic sensors 120 may not be arranged or distributed in an array extending all the way around or completely around the patient 14.

Instead, the electrodes 112 and acoustic sensors 120 may be arranged in an array that extends only part of the way or partially around the patient 14. For example, the electrodes 112 and acoustic sensors 120 may be distributed on the anterior, posterior, and left sides of the patient with less or no electrodes and acoustic sensors proximate the right side (including posterior and anterior regions of the right side of the patient).

The local computing device 140 may record and analyze the torso-surface potential signals sensed by electrodes 112 and the sound signals sensed by the acoustic sensors 120, which are amplified/conditioned by the interface/amplifier circuitry 116. The local computing device 140 may be configured to analyze the electrical signals from the electrodes 112 to provide electrocardiogram (ECG) signals, information, or data from the patient's heart as will be further described herein. The local computing device 140 may be configured to analyze the electrical signals from the acoustic sensors 120 to provide sound signals, information, or data from the patient's body and/or devices implanted therein (such as a left ventricular assist device).

Additionally, the local computing device 140, the remote computing device 160, and the cloud computing device 190 may be configured to provide graphical user interfaces 132, 172 depicting various information related to the electrode apparatus 110 and the data gathered, or sensed, using the electrode apparatus 110. In at least one embodiment, the cloud computing device 190 may provide one or both of the graphical user interfaces 132, 172 to the local computing device 140 and the remote computing device 160. For example, the graphical user interfaces 132, 172 may depict ECGs including P-waves and/or QRS complexes obtained using the electrode apparatus 110 and sound data including sound waves obtained using the acoustic sensors 120 as well as other information related thereto. Illustrative systems, methods, and devices may noninvasively use the electrical information collected using the electrode apparatus 110 and the sound information collected using the acoustic sensors 120 to evaluate a patient's cardiac health and to evaluate and configure cardiac therapy being delivered to the patient.

Further, the electrode apparatus 110 may further include reference electrodes and/or drive electrodes to be, e.g., positioned about the lower torso of the patient 14, that may be further used by the system 100. For example, the electrode apparatus 110 may include three reference electrodes, and the signals from the three reference electrodes may be combined to provide a reference signal. Further, the electrode apparatus 110 may use three caudal reference electrodes (e.g., instead of standard references used in a Wilson Central Terminal) to get a “true” unipolar signal with less noise from averaging three caudally located reference signals.

FIG. 3 illustrates another illustrative electrode apparatus 110 that includes a plurality of electrodes 112 configured to surround the heart of the patient 14 and record, or monitor, the electrical signals associated with the depolarization and repolarization of the heart after the signals have propagated through the torso of the patient 14 and a plurality of acoustic sensors 120 configured to surround the heart of the patient 14 and record, or monitor, the sound signals associated with the heart after the signals have propagated through the torso of the patient 14.

The electrode apparatus 110 may include a vest 114 upon which the plurality of electrodes 112 and the plurality of acoustic sensors 120 may be attached, or to which the electrodes 112 and the acoustic sensors 120 may be coupled. In at least one embodiment, the plurality, or array, of electrodes 112 may be used to collect electrical information such as, e.g., atrial electrical heterogeneity, electrical heterogeneity information, P-wave electrical heterogeneity, surrogate electrical activation times, etc. Similar to the electrode apparatus 110 of FIG. 2, the electrode apparatus 110 of FIG. 3 may include interface/amplifier circuitry 116 electrically coupled to each of the electrodes 112 and the acoustic sensors 120 through a wired connection 118 and be configured to transmit signals from the electrodes 112 and the acoustic sensors 120 to the local computing device 140. As illustrated, the electrodes 112 and the acoustic sensors 120 may be distributed over the torso of a patient 14, including, for example, the posterior, lateral, posterolateral, anterolateral, and anterior locations of the torso of a patient 14.

The vest 114 may be formed of fabric with the electrodes 112 and the acoustic sensors 120 attached to the fabric. The vest 114 may be configured to maintain the position and spacing of electrodes 112 and the acoustic sensors 120 on the torso of the patient 14. Further, the vest 114 may be marked to assist in determining the location of the electrodes 112 and the acoustic sensors 120 on the surface of the torso of the patient 14. In some examples, there may be about 25 to about 256 electrodes 112 and about 25 to about 256 acoustic sensors 120 distributed around the torso of the patient 14, though other configurations may have more or fewer electrodes 112 and more or fewer acoustic sensors 120.

The illustrative systems, methods, devices, and methods may be used to provide noninvasive assistance to configure of cardiac therapy being presently delivered to the patient (e.g., by an implantable medical device, etc.). For example, the illustrative systems, methods, and devices may be used to configure and/or adjust of one or more atrial therapy settings such as, e.g., selection of pacing electrodes, selection of pacing vectors, selection pacing output parameters, selection of pacing timings, etc.

Further, it is to be understood that the computing apparatus, e.g., include the local computing device 140, the remote computing device 160, and the cloud computing device 190, may be operatively coupled in a plurality of different ways so as to perform, or execute, the functionality described herein. For example, in the embodiment depicted, the computing device 140 may be wireless operably coupled to the remote computing device 160 as depicted by the wireless signal lines emanating therebetween. Additionally, as opposed to wireless connections, one or more of the local computing device 140 and the remoting computing device 160 may be operably coupled through one or wired electrical connections. Furthermore, one or both of the local computing device 140 and the remote computing device 160 may be operably coupled (e.g., wirelessly operably coupled, partially-wirelessly coupled, etc.) to the cloud computing device 190 via a network 191 such as, e.g., the internet. In one embodiment, the cloud computing device 190 may perform all or much of the data analysis on the data from the electrode apparatus 110 and output such analysis to one or both of the local computing device 140 and the remote computing device 160. It is be understood that the data processing and analysis described herein may be provide by one or more of the local computing device 140, the remote computing device 160, and the cloud computing device 190. In other words, the processing and analysis may be distributed among the computing apparatus such as the local computing device 140, the remote computing device 160, and the cloud computing device 190. Additionally, the cloud computing device 190 may include, among other things, an electronic medical records (EMR) system or database that may store information from the systems, methods, and apparatus described herein and provide information to such systems, methods, and apparatus. For example, the EMR of the cloud computing device 190 may provide images (e.g., CT images of the patient's heart) to local computing device 140 and the remote computing device 160 to be used with the data provided by the electrode apparatus 110.

An illustrative method 200 of generating atrial electrical heterogeneity, and in particular, P-wave electrical heterogeneity is depicted in FIG. 4. The illustrative method 200 may be generally described to be used in the efficient, noninvasive configuration (e.g., optimization) of atrial pacing therapy and/or evaluation of a patient's condition (e.g., risk stratification, determination of whether cardiac therapy would benefit the patient, etc.). The illustrative method 200 may be described as being noninvasive because the method does not use invasive apparatus to perform the evaluation of pacing therapy. The pacing therapy being delivered, however, may be described as being invasive such as, e.g., one or more pacing electrodes may be implanted proximate a patient's heart. Thus, the illustrative method 200 may be used to evaluate and configure such invasive cardiac therapy.

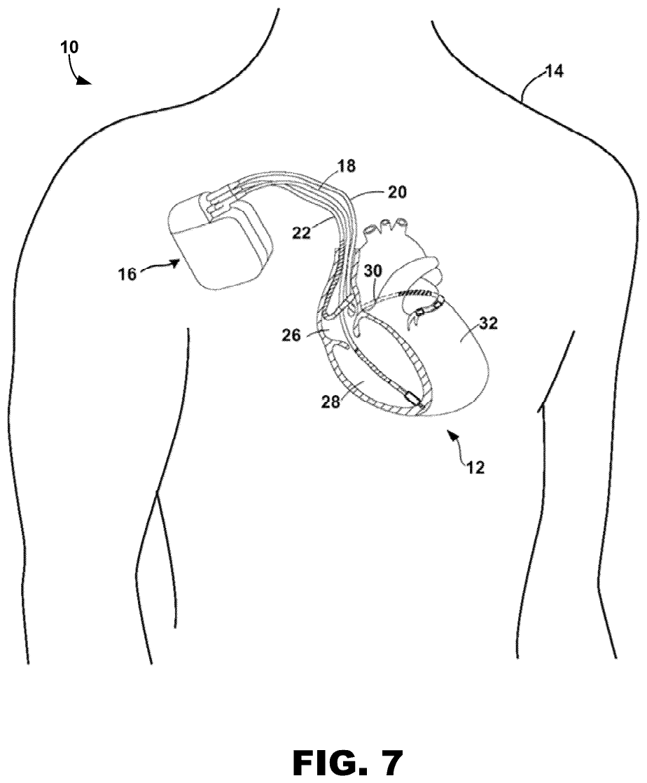

The illustrative method 200 may optionally include delivering atrial pacing therapy 201, e.g., using the devices described herein with respect to FIGS. 7-9. For example, Bachman bundle pacing, atrial septal pacing, left atrial pacing, or biatrial pacing may be delivered to a patient. In other embodiments, the method 200 may be performed when no atrial pacing therapy is being delivered to the patient to, e.g., obtain baseline data or information about the patient's condition prior to delivery of therapy, determine whether the patient would benefit from atrial pacing therapy, provide patient risk stratification, etc.

The method 200 may further include monitoring electrical activity 202 using a plurality of external electrodes. The plurality of external electrodes may be similar to the external electrodes provided by the electrode apparatus 110 as described herein with respect to FIGS. 1-3. For example, the plurality of external electrodes may be part, or incorporated into, a vest or band that is located about a patient's torso. More specifically, the plurality of electrodes may be described as being surface electrodes positioned in an array configured to be located proximate the skin of the torso of a patient. For instance, each of the electrodes may be positioned or located about the torso of the patient so as to monitor electrical activity (e.g., acquire torso-potentials) from a plurality of different locations about the torso of the patient. Each of the different locations where the electrodes are located may correspond to the electrical activity or activation of different portions or regions of cardiac tissue of the patient's heart. Thus, for example, the plurality of electrodes may record, or monitor, the electrical signals associated with the depolarization and repolarization of a plurality of different locations of, or about, the heart after the signals have propagated through the torso of a patient. According to various embodiments, the plurality of external electrodes may include, or comprise, a plurality of anterior electrodes that are located proximate skin of the anterior of the patient's torso, left lateral or left side electrodes that are located proximate skin of the left lateral or left side of the patient's torso, and posterior electrodes that are located proximate skin of the posterior of the patient's torso.

It may be described that, when using a plurality of external electrodes, the monitoring process 202 may provide a plurality of electrocardiograms (ECGs), signals representative of the depolarization and repolarization of the patient's heart. The plurality of ECGs may, in turn, be used to generate atrial electrical heterogeneity 205, such as P-wave electrical heterogeneity, as will be described further herein. The electrical activity may be monitored for a monitoring time period such as 5 seconds or for a selected number of cardiac cycles such as 6 cardiac cycles during a given state of atrial rhythm (e.g., intrinsic or paced)

Prior to generation of atrial electrical heterogeneity 205, the method 200 may filter the monitored electrical activity 204. More specifically, each of the plurality of signals of the monitored electrical activity, each signal corresponding to a different electrode of the plurality of electrode, may be filtered to remove one or more of high frequency noise and noisy or invalid signals due to loss of electrode contact. In one or more embodiments, noisy signals may be removed from the plurality of signals (e.g., removed from the data set). In one or more embodiments, a removed signal may then be regenerated by interpolating proximate signals. Additionally, in one embodiment, the signals may be filtered with 20 Hz Bessel filter to remove high frequency noises, reject electrodes with noise, and/or invalid signals due to loss of contact.

The method 200 may further generating atrial electrical heterogeneity 205, e.g., after optionally filtering the signals. Generating atrial electrical heterogeneity 205 may include windowing the P-wave of each signal 206. In other words, the portion of each signal within which the P-wave is found may be selected for further analysis to generating the atrial electrical heterogeneity. The P-wave may be windowed in a variety of ways.

In one embodiment, a QRS onset may be determined in a signal, and a loopback window prior the QRS onset may be selected thereby providing the P-wave window for each signal. Such analysis may be performed for each signal or the earliest QRS onset may be determined from the plurality of signals and the lookback window may be defined prior the earliest QRS onset. Nonetheless, upon windowing the P-wave, the portion each signal that it utilized for further analysis to generate atrial electrical heterogeneity will have been identified.

The lookback window precedes the QRS onset by between about 150 milliseconds (ms) and about 400 ms. In one embodiment, the lookback window precedes the QRS onset by 300 ms. In one or more embodiments, the lookback window precedes the QRS onset by less than or equal to 400 ms, by less than or equal to 350 ms, by less than or equal to 300 ms, by less than or equal to 250 ms, etc. and/or by greater than or equal to 150 ms, by greater than or equal to 200 ms, etc. Additionally, the end of lookback window may be offset prior to the QRS onset by between about 80 ms and about 200 ms. In this way, the lookback window may define a P-wave onset as being the beginning of the lookback window and P-wave offset as being the end of the lookback window. In another embodiment, a P-wave onset and P-wave offset may be determined without determining a QRS onset through signal analysis.

For each signal of the plurality of signals of the monitored electrical activity, a distribution metric may be generated 208 for the signal within the lookback time window. In this way, a plurality of distribution metrics may be generated, one for each signal. The distribution metric may be any metric or statistic that is representative of the dispersion or distribution of the signal within the lookback window or P-wave window. In one embodiment, the distribution metric is a standard deviation of the signal within the lookback window or P-wave window. In one embodiment, the distribution metric is based on an interquartile deviation of the signal amplitude within the lookback window or P-wave window. The distribution metric may be based on other statistical measures of dispersion such as, but not limited to, mean deviation, mean-squared deviation, etc.

Then, the maximum, peak, or largest, distribution metric of the plurality of generated for distribution metrics for the plurality of signals 210 may be determined or identified resulting in the P-wave electrical heterogeneity. In other words, in this embodiment, the P-wave electrical heterogeneity is the maximum distribution metric of the plurality of generated for distribution metrics.

The method 200 further includes evaluation of the atrial electrical heterogeneity 212, which may be used to evaluate the cardiac therapy such as atrial pacing therapy being delivered during the monitoring of electrical activity or used to evaluate the patient's cardiac condition or state. For example, the atrial electrical heterogeneity may be compared to a threshold value or to a baseline value generated during intrinsic cardiac activation (i.e., no delivery of therapy).

For example, evaluation of the atrial electrical heterogeneity 212 may include determining acceptability of the atrial pacing therapy based on P-wave electrical heterogeneity by comparing the P-wave electrical heterogeneity to a threshold value. When the P-wave electrical heterogeneity is the maximum standard deviation, the threshold value between about 0.01 millivolts (mV) and about 0.2 mV. In one embodiment, the maximum standard deviation is 0.1 mV, and thus, for instance, if the maximum standard deviation is less than or equal to 0.1 mV, then it may be determined that the atrial pacing therapy is acceptable. In one embodiment, the maximum standard deviation is 0.2 mV, and thus, for instance, if the maximum standard deviation is greater than or equal to 0.2 mV, then it may be determined that the patient is undergoing atrial dyssynchrony and/or risk of atrial fibrillation.

Further, for example, evaluation of the atrial electrical heterogeneity 212 may include determining acceptability of atrial pacing therapy based on the P-wave electrical heterogeneity by comparing the P-wave electrical heterogeneity to a baseline P-wave electrical heterogeneity generated from obtained external electrical activity during intrinsic cardiac activation. More specially, in one example, determining acceptability of atrial pacing therapy based on the P-wave electrical heterogeneity may include determining that the atrial pacing therapy is acceptable if the P-wave electrical heterogeneity is less than the baseline P-wave electrical heterogeneity by a comparison percentage. The comparison percentage may between about 10% and about 50%. In one or more embodiments, the comparison percentage may be greater than or equal to 10%, greater than or equal to 20%, greater than or equal to 30%, etc. and/or less than or equal to 50%, less than or equal to 40%, less than or equal to 25%, etc. For instance, if the P-wave electrical heterogeneity is less than about 20% of than the baseline P-wave electrical heterogeneity, then it may be determined that the atrial pacing therapy is acceptable.

Additionally, the generation of atrial electrical heterogeneity 205 and evaluation thereof 212 may be used in closed loop optimization of the atrial pacing therapy. For example, one or atrial pacing settings such as pacing electrode, pacing vector, pacing output parameter (e.g., pulse width, pulse frequency, amplitude, etc.), pacing electrode or lead location, and pacing timing, etc. may be adjusted in response to the evaluation thereof 212 to improve the atrial pacing therapy. For instance, the atrial pacing therapy may be delivered using a plurality of different atrial pacing settings, or configurations, each atrial electrical heterogeneity may be generated and then evaluated for each of the plurality of different atrial pacing settings, or configurations.

From such evaluation, a desired pacing settings or configuration may be determined, e.g., based on the pacing settings or configuration resulting the best atrial electrical heterogeneity, based on the pacing settings or configuration resulting acceptable atrial electrical heterogeneity and providing optical power savings to extend battery life, etc.

Monitored electrical activity 300 during intrinsic activation of a patient is depicted in FIG. 5A. A P-wave window 302 was selected, or identified, from the monitored electrical activity 300, and a maximum, or largest, standard deviation of a plurality of standard deviations of the signals within the P-wave window was 0.14. As such, the P-wave electrical heterogeneity (PEH) is 0.14 for this patient.

Monitored electrical activity 304 during delivery of Bachman bundle pacing to the patient is depicted in FIG. 5B. A P-wave window 306 was selected, or identified, from the monitored electrical activity 304, and a maximum, or largest, standard deviation of a plurality of standard deviations of the signals within the P-wave window was 0.06. As such, the PEH is 0.06 for this patient when receiving Bachman bundle pacing. As a result, delivery of Bachman bundle pacing resulted in a substantial decrease in the PEH from intrinsic activation thereby indicating that the Bachman bundle pacing is acceptable.

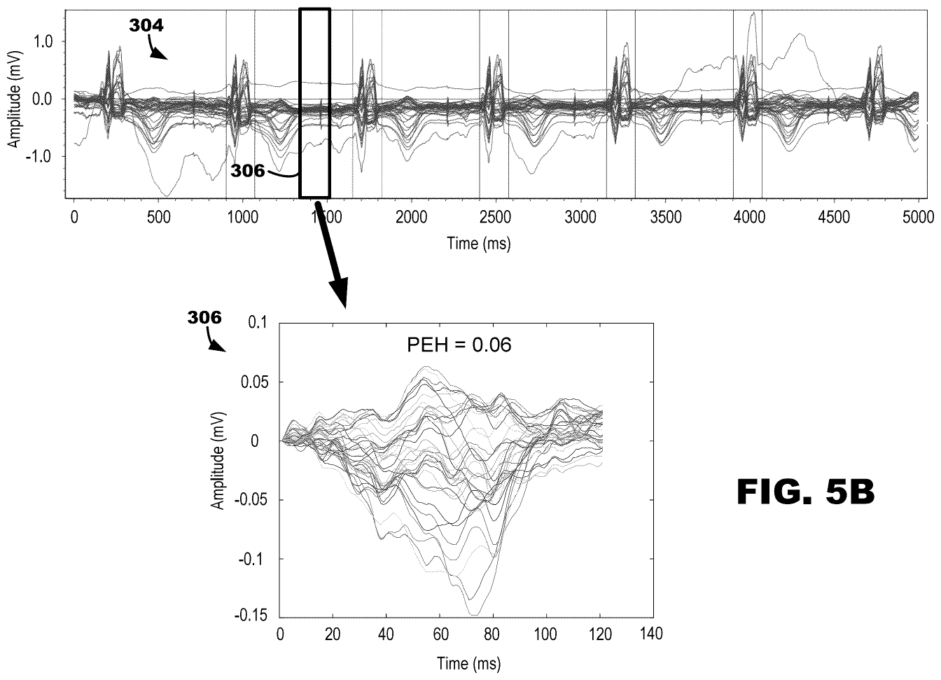

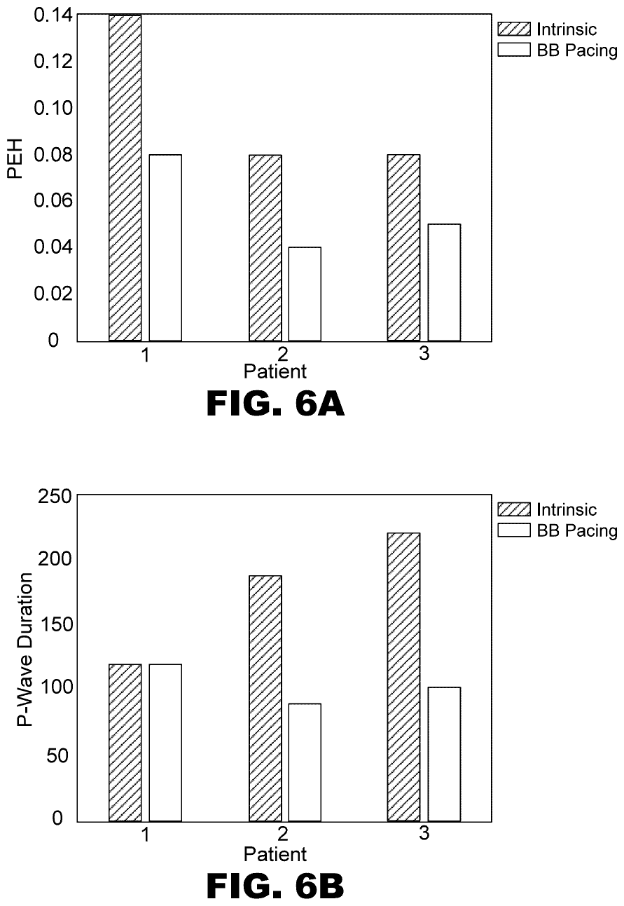

P-wave electrical heterogeneity generated using the systems and methods described herein for three difference patients during intrinsic activation and during Bachman bundle pacing is depicted in FIG. 6A. As shown, the PEH decreased for each patient from intrinsic activation during Bachman bundle pacing, which may indicate acceptable or effective therapy.

P-wave duration for three difference patients during intrinsic activation and during Bachman bundle pacing is depicted in FIG. 6B. As shown, the P-wave duration decreased for patients 2 and 3 from intrinsic activation during Bachman bundle pacing while P-wave duration did not decrease from intrinsic activation during Bachman bundle pacing. As such, P-wave duration may not be as reliable indicator of acceptable or effective Bachman bundle pacing as PEH.

Illustrative cardiac therapy systems and devices may be further described herein with reference to FIGS. 7-9 that may utilize the illustrative systems, interfaces, methods, and processes described herein with respect to FIGS. 1-6. The illustrative cardiac therapy systems and devices described herein may be configured to deliver atrial septal pacing, Bachmann's bundle pacing, left atrial pacing, or biatrial pacing. For example, the Bachmann's bundle may be described as a path for electrical activation of the left atrium during normal sinus rhythm and is therefore considered to be part of the “atrial conduction system” of the heart. Therefore, atrial pacing using a single lead in the atrium that targets the Bachmann's bundle for synchronized atrial activation may be used to treat atrial dyssynchrony. Another possible pacing therapy for treating atrial dyssynchrony may include multi-site atrial stimulation using two leads, one positioned in the right atrium and the other being positioned within the left atrium.

FIG. 7 is a conceptual diagram illustrating an illustrative therapy system 10 that may be used to deliver pacing therapy to a patient 14. Patient 14 may, but not necessarily, be a human. The therapy system 10 may include an implantable medical device 16 (IMD), which may be coupled to leads 18, 20, 22. The IMD 16 may be, e.g., an implantable pacemaker, cardioverter, and/or defibrillator, that delivers, or provides, electrical signals (e.g., paces, etc.) to and/or senses electrical signals from the heart 12 of the patient 14 via electrodes coupled to one or more of the leads 18, 20, 22.

The leads 18, 20, 22 extend into the heart 12 of the patient 14 to sense electrical activity of the heart 12 and/or to deliver electrical stimulation to the heart 12. In the example shown in FIG. 7, the right ventricular (RV) lead 18 extends through one or more veins (not shown), the superior vena cava (not shown), and the right atrium 26, and into the right ventricle 28. The left ventricular (LV) coronary sinus lead 20 extends through one or more veins, the vena cava, the right atrium 26, and into the coronary sinus 30 to a region adjacent to the free wall of the left ventricle 32 of the heart 12. The right atrial (RA) lead 22 extends through one or more veins and the vena cava, and into the right atrium 26 of the heart 12.

The IMD 16 may sense, among other things, electrical signals attendant to the depolarization and repolarization of the heart 12 via electrodes coupled to at least one of the leads 18, 20, 22. In some examples, the IMD 16 provides pacing therapy (e.g., pacing pulses) to the heart 12 based on the electrical signals sensed within the heart 12. The IMD 16 may be operable to adjust one or more parameters associated with the pacing therapy such as, e.g., A-V delay and other various timings, pulse wide, amplitude, voltage, burst length, etc. Further, the IMD 16 may be operable to use various electrode configurations to deliver pacing therapy, which may be unipolar, bipolar, quadripoloar, or further multipolar. For example, a multipolar lead may include several electrodes that can be used for delivering pacing therapy. Hence, a multipolar lead system may provide, or offer, multiple electrical vectors to pace from. A pacing vector may include at least one cathode, which may be at least one electrode located on at least one lead, and at least one anode, which may be at least one electrode located on at least one lead (e.g., the same lead, or a different lead) and/or on the casing, or can, of the IMD. While improvement in cardiac function as a result of the pacing therapy may primarily depend on the cathode, the electrical parameters like impedance, pacing threshold voltage, current drain, longevity, etc. may be more dependent on the pacing vector, which includes both the cathode and the anode. The IMD 16 may also provide defibrillation therapy and/or cardioversion therapy via electrodes located on at least one of the leads 18, 20, 22. Further, the IMD 16 may detect arrhythmia of the heart 12, such as fibrillation of the ventricles 28, 32, and deliver defibrillation therapy to the heart 12 in the form of electrical pulses. In some examples, IMD 16 may be programmed to deliver a progression of therapies, e.g., pulses with increasing energy levels, until a fibrillation of heart 12 is stopped.

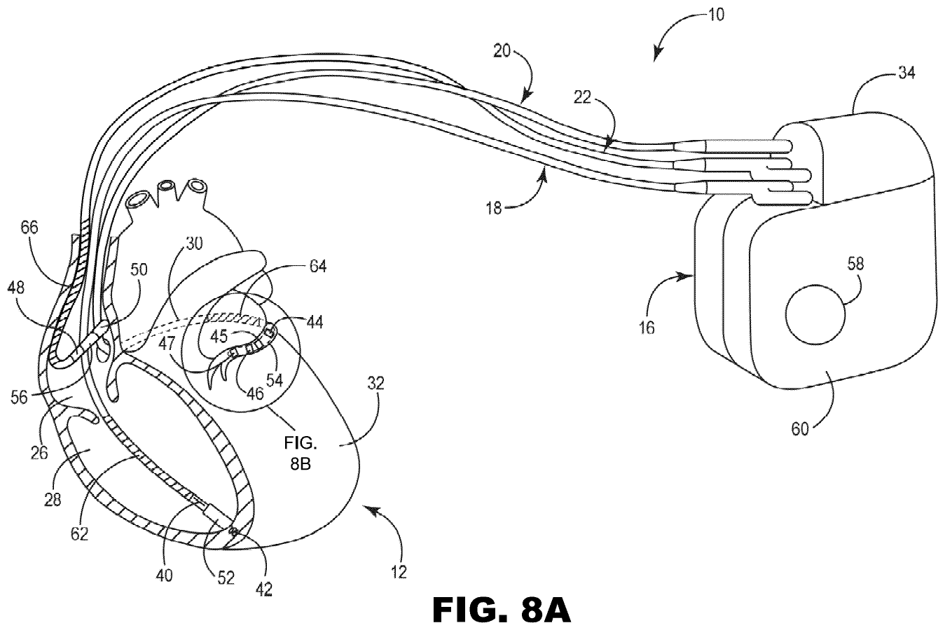

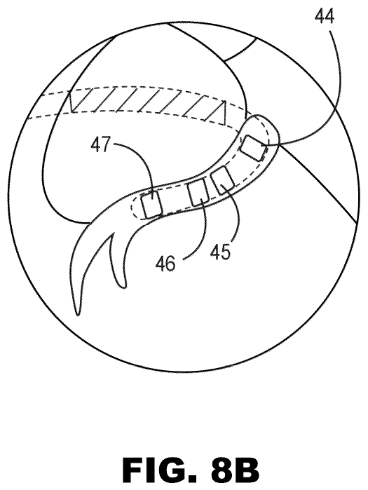

FIGS. 8A-8B are conceptual diagrams illustrating the IMD 16 and the leads 18, 20, 22 of therapy system 10 of FIG. 7 in more detail. The leads 18, 20, 22 may be electrically coupled to a therapy delivery module (e.g., for delivery of pacing therapy), a sensing module (e.g., for sensing one or more signals from one or more electrodes), and/or any other modules of the IMD 16 via a connector block 34. In some examples, the proximal ends of the leads 18, 20, 22 may include electrical contacts that electrically couple to respective electrical contacts within the connector block 34 of the IMD 16. In addition, in some examples, the leads 18, 20, 22 may be mechanically coupled to the connector block 34 with the aid of set screws, connection pins, or another suitable mechanical coupling mechanism.

Each of the leads 18, 20, 22 includes an elongated insulative lead body, which may carry a number of conductors (e.g., concentric coiled conductors, straight conductors, etc.) separated from one another by insulation (e.g., tubular insulative sheaths). In the illustrated example, bipolar electrodes 40, 42 are located proximate to a distal end of the lead 18. In addition, bipolar electrodes 44, 45, 46, 47 are located proximate to a distal end of the lead 20 and bipolar electrodes 48, 50 are located proximate to a distal end of the lead 22.

The electrodes 40, 44, 45, 46, 47, 48 may take the form of ring electrodes, and the electrodes 42, 50 may take the form of extendable helix tip electrodes mounted retractably within the insulative electrode heads 52, 54, 56, respectively. Each of the electrodes 40, 42, 44, 45, 46, 47, 48, 50 may be electrically coupled to a respective one of the conductors (e.g., coiled and/or straight) within the lead body of its associated lead 18, 20, 22, and thereby coupled to a respective one of the electrical contacts on the proximal end of the leads 18, 20, 22.

Additionally, electrodes 44, 45, 46 and 47 may have an electrode surface area of about 5.3 mm² to about 5.8 mm². Electrodes 44, 45, 46, and 47 may also be referred to as LV1, LV2, LV3, and LV4, respectively. The LV electrodes (i.e., left ventricle electrode 1 (LV1) 44, left ventricle electrode 2 (LV2) 45, left ventricle electrode 3 (LV3) 46, and left ventricle 4 (LV4) 47 etc.) on the lead 20 can be spaced apart at variable distances. For example, electrode 44 may be a distance of, e.g., about 21 millimeters (mm), away from electrode 45, electrodes 45 and 46 may be spaced a distance of, e.g., about 1.3 mm to about 1.5 mm, away from each other, and electrodes 46 and 47 may be spaced a distance of, e.g., 20 mm to about 21 mm, away from each other.

The electrodes 40, 42, 44, 45, 46, 47, 48, 50 may further be used to sense electrical signals (e.g., morphological waveforms within electrograms (EGM)) attendant to the depolarization and repolarization of the heart 12. The electrical signals are conducted to the IMD 16 via the respective leads 18, 20, 22. In some examples, the IMD 16 may also deliver pacing pulses via the electrodes 40, 42, 44, 45, 46, 47, 48, 50 to cause depolarization of cardiac tissue of the patient's heart 12. In some examples, as illustrated in FIG. 8A, the IMD 16 includes one or more housing electrodes, such as housing electrode 58, which may be formed integrally with an outer surface of a housing 60 (e.g., hermetically sealed housing) of the IMD 16 or otherwise coupled to the housing 60. Any of the electrodes 40, 42, 44, 45, 46, 47, 48, 50 may be used for unipolar sensing or pacing in combination with the housing electrode 58. It is generally understood by those skilled in the art that other electrodes can also be selected to define, or be used for, pacing and sensing vectors. Further, any of electrodes 40, 42, 44, 45, 46, 47, 48, 50, 58, when not being used to deliver pacing therapy, may be used to sense electrical activity during pacing therapy.

As described in further detail with reference to FIG. 8A, the housing 60 may enclose a therapy delivery module that may include a stimulation generator for generating cardiac pacing pulses and defibrillation or cardioversion shocks, as well as a sensing module for monitoring the electrical signals of the patient's heart (e.g., the patient's heart rhythm). The leads 18, 20, 22 may also include elongated electrodes 62, 64, 66, respectively, which may take the form of a coil. The IMD 16 may deliver defibrillation shocks to the heart 12 via any combination of the elongated electrodes 62, 64, 66 and the housing electrode 58. The electrodes 58, 62, 64, 66 may also be used to deliver cardioversion pulses to the heart 12. Further, the electrodes 62, 64, 66 may be fabricated from any suitable electrically conductive material, such as, but not limited to, platinum, platinum alloy, and/or other materials known to be usable in implantable defibrillation electrodes. Since electrodes 62, 64, 66 are not generally configured to deliver pacing therapy, any of electrodes 62, 64, 66 may be used to sense electrical activity and may be used in combination with any of electrodes 40, 42, 44, 45, 46, 47, 48, 50, 58. In at least one embodiment, the RV elongated electrode 62 may be used to sense electrical activity of a patient's heart during the delivery of pacing therapy (e.g., in combination with the housing electrode 58, or defibrillation electrode-to-housing electrode vector).

The configuration of the illustrative therapy system 10 illustrated in FIGS. 7-9 is merely one example. In other examples, the therapy system may include epicardial leads and/or patch electrodes instead of or in addition to the transvenous leads 18, 20, 22 illustrated in FIG. 7. Additionally, in other examples, the therapy system 10 may be implanted in/around the cardiac space without transvenous leads (e.g., leadless/wireless pacing systems) or with leads implanted (e.g., implanted transvenously or using approaches) into the left chambers of the heart (in addition to or replacing the transvenous leads placed into the right chambers of the heart as illustrated in FIG. 7). Further, in one or more embodiments, the IMD 16 need not be implanted within the patient 14. For example, the IMD 16 may deliver various cardiac therapies to the heart 12 via percutaneous leads that extend through the skin of the patient 14 to a variety of positions within or outside of the heart 12. In one or more embodiments, the system 10 may utilize wireless pacing (e.g., using energy transmission to the intracardiac pacing component(s) via ultrasound, inductive coupling, RF, etc.) and sensing cardiac activation using electrodes on the can/housing and/or on subcutaneous leads.

In other examples of therapy systems that provide electrical stimulation therapy to the heart 12, such therapy systems may include any suitable number of leads coupled to the IMD 16, and each of the leads may extend to any location within or proximate to the heart 12. For example, other examples of therapy systems may include three transvenous leads located as illustrated in FIGS. 7-9. Still further, other therapy systems may include a single lead that extends from the IMD 16 into the right atrium 26 or the right ventricle 28, or two leads that extend into a respective one of the right atrium 26 and the right ventricle 28.

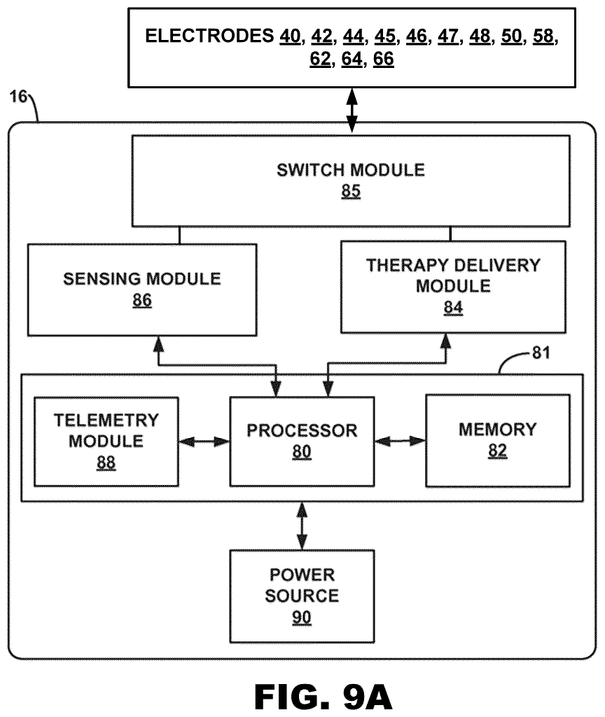

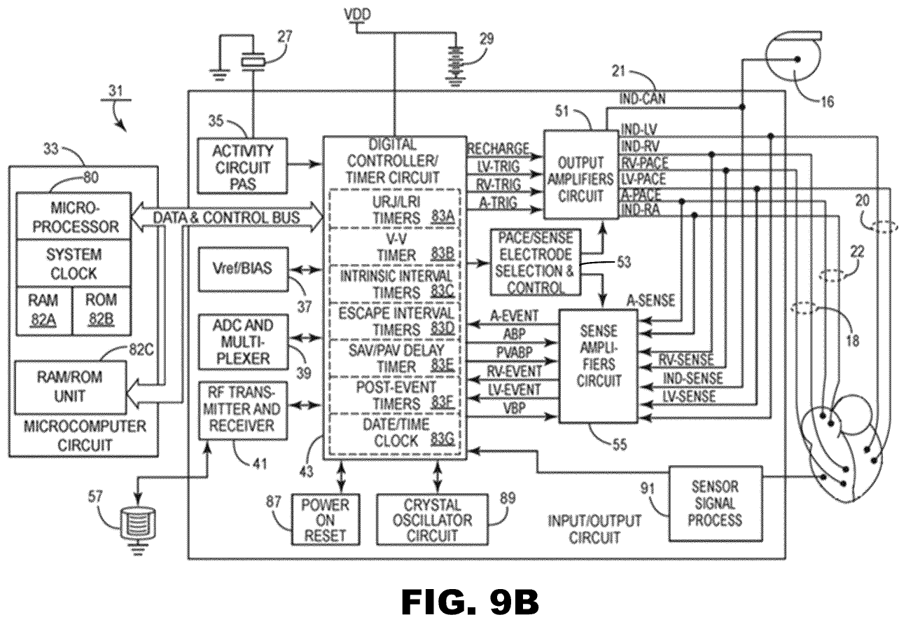

FIG. 9A is a functional block diagram of one illustrative configuration of the IMD 16. As shown, the IMD 16 may include a control module 81, a therapy delivery module 84 (e.g., which may include a stimulation generator), a sensing module 86, and a power source 90.

The control module, or apparatus, 81 may include a processor 80, memory 82, and a telemetry module, or apparatus, 88. The memory 82 may include computer-readable instructions that, when executed, e.g., by the processor 80, cause the IMD 16 and/or the control module 81 to perform various functions attributed to the IMD 16 and/or the control module 81 described herein. Further, the memory 82 may include any volatile, non-volatile, magnetic, optical, and/or electrical media, such as a random-access memory (RAM), read-only memory (ROM), non-volatile RAM (NVRAM), electrically erasable programmable ROM (EEPROM), flash memory, and/or any other digital media. An illustrative capture management module may be the left ventricular capture management (LVCM) module described in U.S. Pat. No. 7,684,863 entitled “LV THRESHOLD MEASUREMENT AND CAPTURE MANAGEMENT” and issued Mar. 23, 2010, which is incorporated herein by reference in its entirety.

The processor 80 of the control module 81 may include any one or more of a microprocessor, a controller, a digital signal processor (DSP), an application specific integrated circuit (ASIC), a field-programmable gate array (FPGA), and/or equivalent discrete or integrated logic circuitry. In some examples, the processor 80 may include multiple components, such as any combination of one or more microprocessors, one or more controllers, one or more DSPs, one or more ASICs, and/or one or more FPGAs, as well as other discrete or integrated logic circuitry. The functions attributed to the processor 80 herein may be embodied as software, firmware, hardware, or any combination thereof.

The control module 81 may control the therapy delivery module 84 to deliver therapy (e.g., electrical stimulation therapy such as pacing) to the heart 12 according to a selected one or more therapy programs, which may be stored in the memory 82. More, specifically, the control module 81 (e.g., the processor 80) may control various parameters of the electrical stimulus delivered by the therapy delivery module 84 such as, e.g., A-V delays, V-V delays, pacing pulses with the amplitudes, pulse widths, frequency, or electrode polarities, etc., which may be specified by one or more selected therapy programs (e.g., A-V and/or V-V delay adjustment programs, pacing therapy programs, pacing recovery programs, capture management programs, etc.). As shown, the therapy delivery module 84 is electrically coupled to electrodes 40, 42, 44, 45, 46, 47, 48, 50, 58, 62, 64, 66, e.g., via conductors of the respective lead 18, 20, 22, or, in the case of housing electrode 58, via an electrical conductor disposed within housing 60 of IMD 16. Therapy delivery module 84 may be configured to generate and deliver electrical stimulation therapy such as pacing therapy to the heart 12 using one or more of the electrodes 40, 42, 44, 45, 46, 47, 48, 50, 58, 62, 64, 66.

For example, therapy delivery module 84 may deliver pacing stimulus (e.g., pacing pulses) via ring electrodes 40, 44, 45, 46, 47, 48 coupled to leads 18, 20, 22 and/or helical tip electrodes 42, 50 of leads 18, 22. Further, for example, therapy delivery module 84 may deliver defibrillation shocks to heart 12 via at least two of electrodes 58, 62, 64, 66. In some examples, therapy delivery module 84 may be configured to deliver pacing, cardioversion, or defibrillation stimulation in the form of electrical pulses. In other examples, therapy delivery module 84 may be configured deliver one or more of these types of stimulation in the form of other signals, such as sine waves, square waves, and/or other substantially continuous time signals.

The IMD 16 may further include a switch module 85 and the control module 81 (e.g., the processor 80) may use the switch module 85 to select, e.g., via a data/address bus, which of the available electrodes are used to deliver therapy such as pacing pulses for pacing therapy, or which of the available electrodes are used for sensing. The switch module 85 may include a switch array, switch matrix, multiplexer, or any other type of switching device suitable to selectively couple the sensing module 86 and/or the therapy delivery module 84 to one or more selected electrodes. More specifically, the therapy delivery module 84 may include a plurality of pacing output circuits. Each pacing output circuit of the plurality of pacing output circuits may be selectively coupled, e.g., using the switch module 85, to one or more of the electrodes 40, 42, 44, 45, 46, 47, 48, 50, 58, 62, 64, 66 (e.g., a pair of electrodes for delivery of therapy to a bipolar or multipolar pacing vector). In other words, each electrode can be selectively coupled to one of the pacing output circuits of the therapy delivery module using the switching module 85.

The sensing module 86 is coupled (e.g., electrically coupled) to sensing apparatus, which may include, among additional sensing apparatus, the electrodes 40, 42, 44, 45, 46, 47, 48, 50, 58, 62, 64, 66 to monitor electrical activity of the heart 12, e.g., electrocardiogram (ECG)/electrogram (EGM) signals, etc. The ECG/EGM signals may be used to measure or monitor activation times (e.g., ventricular activations times, etc.), heart rate (HR), heart rate variability (HRV), heart rate turbulence (HRT), deceleration/acceleration capacity, deceleration sequence incidence, T-wave alternans (TWA), P-wave to P-wave intervals (also referred to as the P-P intervals or A-A intervals), R-wave to R-wave intervals (also referred to as the R-R intervals or V-V intervals), P-wave to QRS complex intervals (also referred to as the P-R intervals, A-V intervals, or P-Q intervals), QRS-complex morphology, ST segment (i.e., the segment that connects the QRS complex and the T-wave), T-wave changes, QT intervals, electrical vectors, etc.

The switch module 85 may also be used with the sensing module 86 to select which of the available electrodes are used, or enabled, to, e.g., sense electrical activity of the patient's heart (e.g., one or more electrical vectors of the patient's heart using any combination of the electrodes 40, 42, 44, 45, 46, 47, 48, 50, 58, 62, 64, 66). Likewise, the switch module 85 may also be used with the sensing module 86 to select which of the available electrodes are not to be used (e.g., disabled) to, e.g., sense electrical activity of the patient's heart (e.g., one or more electrical vectors of the patient's heart using any combination of the electrodes 40, 42, 44, 45, 46, 47, 48, 50, 58, 62, 64, 66), etc. In some examples, the control module 81 may select the electrodes that function as sensing electrodes via the switch module within the sensing module 86, e.g., by providing signals via a data/address bus.

In some examples, sensing module 86 includes a channel that includes an amplifier with a relatively wider pass band than the R-wave or P-wave amplifiers. Signals from the selected sensing electrodes may be provided to a multiplexer, and thereafter converted to multi-bit digital signals by an analog-to-digital converter for storage in memory 82, e.g., as an electrogram (EGM). In some examples, the storage of such EGMs in memory 82 may be under the control of a direct memory access circuit.

In some examples, the control module 81 may operate as an interrupt-driven device and may be responsive to interrupts from pacer timing and control module, where the interrupts may correspond to the occurrences of sensed P-waves and R-waves and the generation of cardiac pacing pulses. Any necessary mathematical calculations may be performed by the processor 80 and any updating of the values or intervals controlled by the pacer timing and control module may take place following such interrupts. A portion of memory 82 may be configured as a plurality of recirculating buffers, capable of holding one or more series of measured intervals, which may be analyzed by, e.g., the processor 80 in response to the occurrence of a pace or sense interrupt to determine whether the patient's heart 12 is presently exhibiting atrial or ventricular tachyarrhythmia.

The telemetry module 88 of the control module 81 may include any suitable hardware, firmware, software, or any combination thereof for communicating with another device, such as a programmer. For example, under the control of the processor 80, the telemetry module 88 may receive downlink telemetry from and send uplink telemetry to a programmer with the aid of an antenna, which may be internal and/or external. The processor 80 may provide the data to be uplinked to a programmer and the control signals for the telemetry circuit within the telemetry module 88, e.g., via an address/data bus. In some examples, the telemetry module 88 may provide received data to the processor 80 via a multiplexer.

The various components of the IMD 16 are further coupled to a power source 90, which may include a rechargeable or non-rechargeable battery. A non-rechargeable battery may be selected to last for several years, while a rechargeable battery may be inductively charged from an external device, e.g., on a daily or weekly basis.

FIG. 9B is another embodiment of a functional block diagram for IMD 16 that depicts bipolar RA lead 22, bipolar RV lead 18, and bipolar LV CS lead 20 without the LA CS pace/sense electrodes and coupled with an implantable pulse generator (IPG) circuit 31 having programmable modes and parameters of a bi-ventricular DDD/R type known in the pacing art.

In turn, the sensor signal processing circuit 91 indirectly couples to the timing circuit 43 and via data and control bus to microcomputer circuitry 33. The IPG circuit 31 is illustrated in a functional block diagram divided generally into a microcomputer circuit 33 and a pacing circuit 21. The pacing circuit 21 includes the digital controller/timer circuit 43, the output amplifiers circuit 51, the sense amplifiers circuit 55, the RF telemetry transceiver 41, the activity sensor circuit 35 as well as a number of other circuits and components described below.

Crystal oscillator circuit 89 provides the basic timing clock for the pacing circuit 21 while battery 29 provides power. Power-on-reset circuit 87 responds to initial connection of the circuit to the battery for defining an initial operating condition and similarly, resets the operative state of the device in response to detection of a low battery condition. Reference mode circuit 37 generates stable voltage reference and currents for the analog circuits within the pacing circuit 21. Analog-to-digital converter (ADC) and multiplexer circuit 39 digitize analog signals and voltage to provide, e.g., real time telemetry of cardiac signals from sense amplifiers 55 for uplink transmission via RF transmitter and receiver circuit 41. Voltage reference and bias circuit 37, ADC and multiplexer 39, power-on-reset circuit 87, and crystal oscillator circuit 89 may correspond to any of those used in illustrative implantable cardiac pacemakers.

If the IPG is programmed to a rate responsive mode, the signals output by one or more physiologic sensors are employed as a rate control parameter (RCP) to derive a physiologic escape interval. For example, the escape interval is adjusted proportionally to the patient's activity level developed in the patient activity sensor (PAS) circuit 35 in the depicted, illustrative IPG circuit 31. The patient activity sensor 27 is coupled to the IPG housing and may take the form of a piezoelectric crystal transducer. The output signal of the patient activity sensor 27 may be processed and used as an RCP. Sensor 27 generates electrical signals in response to sensed physical activity that are processed by activity circuit 35 and provided to digital controller/timer circuit 43. Activity circuit 35 and associated sensor 27 may correspond to the circuitry disclosed in U.S. Pat. No. 5,052,388 entitled “METHOD AND APPARATUS FOR IMPLEMENTING ACTIVITY SENSING IN A PULSE GENERATOR” and issued on Oct. 1, 1991, and U.S. Pat. No. 4,428,378 entitled “RATE ADAPTIVE PACER” and issued on Jan. 31, 1984, each of which is incorporated herein by reference in its entirety.

Similarly, the illustrative systems, apparatus, and methods described herein may be practiced in conjunction with alternate types of sensors such as oxygenation sensors, pressure sensors, pH sensors, and respiration sensors, for use in providing rate responsive pacing capabilities.

Alternately, QT time may be used as a rate indicating parameter, in which case no extra sensor is required. Similarly, the illustrative embodiments described herein may also be practiced in non-rate responsive pacemakers.

Data transmission to and from the external programmer is accomplished by way of the telemetry antenna 57 and an associated RF transceiver 41, which serves both to demodulate received downlink telemetry and to transmit uplink telemetry. Uplink telemetry capabilities may include the ability to transmit stored digital information, e.g., operating modes and parameters, EGM histograms, and other events, as well as real time EGMs of atrial and/or ventricular electrical activity and marker channel pulses indicating the occurrence of sensed and paced depolarizations in the atrium and ventricle.

Microcomputer 33 contains a microprocessor 80 and associated system clock and on-processor RAM and ROM chips 82A and 82B, respectively. In addition, microcomputer circuit 33 includes a separate RAM/ROM chip 82C to provide additional memory capacity.

Microprocessor 80 normally operates in a reduced power consumption mode and is interrupt driven. Microprocessor 80 is awakened in response to defined interrupt events, which may include A-TRIG, RV-TRIG, LV-TRIG signals generated by timers in digital timer/controller circuit 43 and A-EVENT, RV-EVENT, and LV-EVENT signals generated by sense amplifiers circuit 55, among others. The specific values of the intervals and delays timed out by digital controller/timer circuit 43 are controlled by the microcomputer circuit 33 by way of data and control bus from programmed-in parameter values and operating modes. In addition, if programmed to operate as a rate responsive pacemaker, a timed interrupt, e.g., every cycle or every two seconds, may be provided in order to allow the microprocessor to analyze the activity sensor data and update the basic A-A, V-A, or V-V escape interval, as applicable. In addition, the microprocessor 80 may also serve to define variable, operative A-V delay intervals, V-V delay intervals, and the energy delivered to each ventricle and/or atrium.

In one embodiment, microprocessor 80 is a custom microprocessor adapted to fetch and execute instructions stored in RAM/ROM unit 82 in a conventional manner. It is contemplated, however, that other implementations may be suitable to practice the present disclosure. For example, an off-the-shelf, commercially available microprocessor or microcontroller, or custom application-specific, hardwired logic, or state-machine type circuit may perform the functions of microprocessor 80.

Digital controller/timer circuit 43 operates under the general control of the microcomputer 33 to control timing and other functions within the pacing circuit 21 and includes a set of timing and associated logic circuits of which certain ones pertinent to the present disclosure are depicted. The depicted timing circuits include URI/LRI timers 83A, V-V delay timer 83B, intrinsic interval timers 83C for timing elapsed V-EVENT to V-EVENT intervals or V-EVENT to A-EVENT intervals or the V-V conduction interval, escape interval timers 83D for timing A-A, V-A, and/or V-V pacing escape intervals, an A-V delay interval timer 83E for timing the A-LVp delay (or A-RVp delay) from a preceding A-EVENT or A-TRIG, a post-ventricular timer 83F for timing post-ventricular time periods, and a date/time clock 83G.

The A-V delay interval timer 83E is loaded with an appropriate delay interval for one ventricular chamber (e.g., either an A-RVp delay or an A-LVp) to time-out starting from a preceding A-PACE or A-EVENT. The interval timer 83E triggers pacing stimulus delivery and can be based on one or more prior cardiac cycles (or from a data set empirically derived for a given patient).

The post-event timer 83F times out the post-ventricular time period following an RV-EVENT or LV-EVENT or a RV-TRIG or LV-TRIG and post-atrial time periods following an A-EVENT or A-TRIG. The durations of the post-event time periods may also be selected as programmable parameters stored in the microcomputer 33. The post-ventricular time periods include the PVARP, a post-atrial ventricular blanking period (PAVBP), a ventricular blanking period (VBP), a post-ventricular atrial blanking period (PVARP) and a ventricular refractory period (VRP) although other periods can be suitably defined depending, at least in part, on the operative circuitry employed in the pacing engine. The post-atrial time periods include an atrial refractory period (ARP) during which an A-EVENT is ignored for the purpose of resetting any A-V delay, and an atrial blanking period (ABP) during which atrial sensing is disabled. It should be noted that the starting of the post-atrial time periods and the A-V delays can be commenced substantially simultaneously with the start or end of each A-EVENT or A-TRIG or, in the latter case, upon the end of the A-PACE which may follow the A-TRIG. Similarly, the starting of the post-ventricular time periods and the V-A escape interval can be commenced substantially simultaneously with the start or end of the V-EVENT or V-TRIG or, in the latter case, upon the end of the V-PACE which may follow the V-TRIG. The microprocessor 80 also optionally calculates A-V delays, V-V delays, post-ventricular time periods, and post-atrial time periods that vary with the sensor-based escape interval established in response to the RCP(s) and/or with the intrinsic atrial and/or ventricular rate.

The output amplifiers circuit 51 contains a RA pace pulse generator (and a LA pace pulse generator if LA pacing is provided), a RV pace pulse generator, a LV pace pulse generator, and/or any other pulse generator configured to provide atrial and ventricular pacing. In order to trigger generation of an RV-PACE or LV-PACE pulse, digital controller/timer circuit 43 generates the RV-TRIG signal at the time-out of the A-RVp delay (in the case of RV pre-excitation) or the LV-TRIG at the time-out of the A-LVp delay (in the case of LV pre-excitation) provided by A-V delay interval timer 83E (or the V-V delay timer 83B). Similarly, digital controller/timer circuit 43 generates an RA-TRIG signal that triggers output of an RA-PACE pulse (or an LA-TRIG signal that triggers output of an LA-PACE pulse, if provided) at the end of the V-A escape interval timed by escape interval timers 83D.

The output amplifiers circuit 51 includes switching circuits for coupling selected pace electrode pairs from among the lead conductors and the IND-CAN electrode to the RA pace pulse generator (and LA pace pulse generator if provided), RV pace pulse generator and LV pace pulse generator. Pace/sense electrode pair selection and control circuit 53 selects lead conductors and associated pace electrode pairs to be coupled with the atrial and ventricular output amplifiers within output amplifiers circuit 51 for accomplishing RA, LA, RV and LV pacing.

The sense amplifiers circuit 55 contains sense amplifiers for atrial and ventricular pacing and sensing. High impedance P-wave and R-wave sense amplifiers may be used to amplify a voltage difference signal that is generated across the sense electrode pairs by the passage of cardiac depolarization wavefronts. The high impedance sense amplifiers use high gain to amplify the low amplitude signals and rely on pass band filters, time domain filtering and amplitude threshold comparison to discriminate a P-wave or R-wave from background electrical noise. Digital controller/timer circuit 43 controls sensitivity settings of the atrial and ventricular sense amplifiers 55.

The sense amplifiers may be uncoupled from the sense electrodes during the blanking periods before, during, and after delivery of a pace pulse to any of the pace electrodes of the pacing system to avoid saturation of the sense amplifiers. The sense amplifiers circuit 55 includes blanking circuits for uncoupling the selected pairs of the lead conductors and the IND-CAN electrode from the inputs of the RA sense amplifier (and LA sense amplifier if provided), RV sense amplifier and LV sense amplifier during the ABP, PVABP and VBP. The sense amplifiers circuit 55 also includes switching circuits for coupling selected sense electrode lead conductors and the IND-CAN electrode to the RA sense amplifier (and LA sense amplifier if provided), RV sense amplifier and LV sense amplifier. Again, sense electrode selection and control circuit 53 selects conductors and associated sense electrode pairs to be coupled with the atrial and ventricular sense amplifiers within the output amplifiers circuit 51 and sense amplifiers circuit 55 for accomplishing RA, LA, RV, and LV sensing along desired unipolar and bipolar sensing vectors.

Right atrial depolarizations or P-waves in the RA-SENSE signal that are sensed by the RA sense amplifier result in a RA-EVENT signal that is communicated to the digital controller/timer circuit 43. Similarly, left atrial depolarizations or P-waves in the LA-SENSE signal that are sensed by the LA sense amplifier, if provided, result in a LA-EVENT signal that is communicated to the digital controller/timer circuit 43. Ventricular depolarizations or R-waves in the RV-SENSE signal are sensed by a ventricular sense amplifier result in an RV-EVENT signal that is communicated to the digital controller/timer circuit 43. Similarly, ventricular depolarizations or R-waves in the LV-SENSE signal are sensed by a ventricular sense amplifier result in an LV-EVENT signal that is communicated to the digital controller/timer circuit 43. The RV-EVENT, LV-EVENT, and RA-EVENT, LA-SENSE signals may be refractory or non-refractory and can inadvertently be triggered by electrical noise signals or aberrantly conducted depolarization waves rather than true R-waves or P-waves.

The techniques described in this disclosure, including those attributed to the IMD 16, the local computing device 140, and/or various constituent components, may be implemented, at least in part, in hardware, software, firmware, or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices, or other devices. The term “module,” “processor,” or “processing circuitry” may generally refer to any of the foregoing logic circuitry, alone or in combination with other logic circuitry, or any other equivalent circuitry.

Such hardware, software, and/or firmware may be implemented within the same device or within separate devices to support the various operations and functions described in this disclosure. In addition, any of the described units, modules, or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed by processing circuitry and/or one or more processors to support one or more aspects of the functionality described in this disclosure.

### ILLUSTRATIVE EXAMPLES

Example Ex1: A system comprising:
a plurality of external electrodes to be located proximate the skin of the torso of a patient to measure electrical activity from tissue of the patient; and
a computing apparatus operably coupled to the plurality of external electrodes and comprising processing circuitry and configured to:
   obtain external electrical activity measured from tissue of a patient; and
   generate a P-wave electrical heterogeneity based on the obtained electrical activity.

Example Ex2: A method comprising:
obtaining external electrical activity measured from tissue of a patient using a plurality of external electrodes; and
generating a P-wave electrical heterogeneity based on the obtained electrical activity.

Example Ex3: The system as in example Ex1 or method as in example Ex2, wherein the electrical activity comprises a plurality of cardiac signals, and wherein generating the P-wave electrical heterogeneity based on the obtained electrical activity comprises:
determining a QRS onset within the plurality of cardiac signals; and
generating a distribution metric for each of the plurality of cardiac signals within a lookback time window resulting in a plurality of distribution metrics, wherein the lookback time window precedes the QRS onset.

Example Ex4: The system or method as in as in example Ex3, wherein the lookback time window is less than or equal to 200 milliseconds.

Example Ex5: The system or method as in any one of examples Ex3-Ex4, wherein generating a P-wave electrical heterogeneity based on the obtained electrical activity further comprises selecting a maximum distribution metric from the plurality of distribution metrics.

Example Ex6: The system or method as in any one of examples Ex3-Ex5, wherein the distribution metric comprises a standard deviation.

Example Ex6: The system or method as in any one of examples Ex1-Ex5, wherein the external electrical activity is measured from tissue of the patient using the plurality of external electrodes during delivery of atrial pacing therapy, wherein the system is further configured to execute or the method further comprises determining acceptability of the atrial pacing therapy based on the generated P-wave electrical heterogeneity.

Example Ex7: The system or method as in example Ex6, wherein determining acceptability of the atrial pacing therapy based on the P-wave electrical heterogeneity comprises comparing the P-wave electrical heterogeneity to a threshold value.

Example Ex8: The system or method as in example Ex7, wherein the threshold value is less than or equal to 0.1 mV.

Example Ex9: The system or method as in example Ex6, wherein the system is further configured to execute or the method further comprises obtaining external electrical activity measured from tissue of a patient during intrinsic cardiac activation, wherein determining acceptability of the atrial pacing therapy based on the P-wave electrical heterogeneity comprises comparing the P-wave electrical heterogeneity to a baseline P-wave electrical heterogeneity generated from obtained external electrical activity during intrinsic cardiac activation.

Example Ex10: The system or method as in example Ex9, wherein determining acceptability of the atrial pacing therapy based on the P-wave electrical heterogeneity comprises determining that the atrial pacing therapy is acceptable if the P-wave electrical heterogeneity is less than the baseline P-wave electrical heterogeneity by a comparison percentage.

Example Ex11: The system or method as in example Ex10, wherein the comparison percentage is less than or equal to 20%.

Example Ex12: The system or method as in example Ex1-Ex11, wherein the system is further configured to execute or the method further comprises filtering the obtained external electrical activity to remove one or more of high frequency noise and noisy or invalid signals due to loss of electrode contact.

This disclosure has been provided with reference to illustrative embodiments and is not meant to be construed in a limiting sense. As described previously, one skilled in the art will recognize that other various illustrative applications may use the techniques as described herein to take advantage of the beneficial characteristics of the apparatus and methods described herein. Various modifications of the illustrative embodiments, as well as additional embodiments of the disclosure, will be apparent upon reference to this description.

What is claimed is:

1. A system comprising:
a plurality of external electrodes to be located proximate the skin of the torso of a patient to measure electrical activity from tissue of the patient; and
a computing apparatus operably coupled to the plurality of external electrodes and comprising processing circuitry and configured to:
obtain external electrical activity measured from tissue of a patient; and
generate a P-wave electrical heterogeneity based on the obtained electrical activity.

2. The system as in claim 1, wherein the electrical activity comprises a plurality of cardiac signals, and wherein generating the P-wave electrical heterogeneity based on the obtained electrical activity comprises:
determining a QRS onset within the plurality of cardiac signals; and
generating a distribution metric for each of the plurality of cardiac signals within a lookback time window resulting in a plurality of distribution metrics, wherein the lookback time window precedes the QRS onset.

3. The system as in claim 2, wherein the lookback time window is less than or equal to 200 milliseconds.

4. The system as in claim 1, wherein generating a P-wave electrical heterogeneity based on the obtained electrical activity further comprises selecting a maximum distribution metric from the plurality of distribution metrics.

5. The system as in claim 1, wherein the distribution metric comprises a standard deviation.

6. The system as in claim 1, wherein the external electrical activity is measured from tissue of the patient using the plurality of external electrodes during delivery of atrial pacing therapy, wherein the computing apparatus is further configured to determine acceptability of the atrial pacing therapy based on the generated P-wave electrical heterogeneity.

7. The system as in claim 6, wherein determining acceptability of the atrial pacing therapy based on the P-wave electrical heterogeneity comprises comparing the P-wave electrical heterogeneity to a threshold value.

8. The system as in claim 7, wherein the threshold value is less than or equal to 0.1 mV.

9. The system as in claim 6, wherein the computing apparatus is further configured to obtain external electrical activity measured from tissue of a patient during intrinsic cardiac activation,
wherein determining acceptability of the atrial pacing therapy based on the P-wave electrical heterogeneity comprises comparing the P-wave electrical heterogeneity to a baseline P-wave electrical heterogeneity generated from obtained external electrical activity during intrinsic cardiac activation.

10. The system as in claim 9, wherein determining acceptability of the atrial pacing therapy based on the P-wave electrical heterogeneity comprises determining that the atrial pacing therapy is acceptable if the P-wave electrical heterogeneity is less than the baseline P-wave electrical heterogeneity by a comparison percentage.

11. The system as in claim 10, wherein the comparison percentage is less than or equal to 20%.

12. The system as claim 1, wherein the computing apparatus is further configured to filter the obtained external electrical activity to remove one or more of high frequency noise and noisy or invalid signals due to loss of electrode contact.

13. A method comprising:
obtaining external electrical activity measured from tissue of a patient using a plurality of external electrodes; and
generating a P-wave electrical heterogeneity based on the obtained electrical activity.

14. The method as in claim 13, wherein the electrical activity comprises a plurality of cardiac signals, and wherein generating the P-wave electrical heterogeneity based on the obtained electrical activity comprises:
determining a QRS onset within the plurality of cardiac signals; and
generating a distribution metric for each of the plurality of cardiac signals within a lookback time window resulting in a plurality of distribution metrics, wherein the lookback time window precedes the QRS onset.

15. The method as in claim 13, wherein generating a P-wave electrical heterogeneity based on the obtained electrical activity further comprises selecting a maximum distribution metric from the plurality of distribution metrics.

16. The method as in claim 1, wherein the external electrical activity is measured from tissue of the patient using the plurality of external electrodes during delivery of atrial pacing therapy, wherein the method further comprises determining acceptability of the atrial pacing therapy based on the generated P-wave electrical heterogeneity.

17. The method as in claim 16, wherein determining acceptability of the atrial pacing therapy based on the P-wave electrical heterogeneity comprises comparing the P-wave electrical heterogeneity to a threshold value.

18. The method as in claim 16, wherein the method further comprises obtaining external electrical activity measured from tissue of a patient during intrinsic cardiac activation,
wherein determining acceptability of the atrial pacing therapy based on the P-wave electrical heterogeneity comprises comparing the P-wave electrical heterogeneity to a baseline P-wave electrical heterogeneity generated from obtained external electrical activity during intrinsic cardiac activation.

19. The method as in claim 18, wherein determining acceptability of the atrial pacing therapy based on the P-wave electrical heterogeneity comprises determining that the atrial pacing therapy is acceptable if the P-wave electrical heterogeneity is less than the baseline P-wave electrical heterogeneity by 20% or less.

20. The method as in claim 13, wherein the method further comprises filtering the obtained external electrical activity to remove one or more of high frequency noise and noisy or invalid signals due to loss of electrode contact.
